(19) United States
(12) Patent Application Publication
(10) Pub. No.: US 2023/0126310 A1
(43) Pub. Date: Apr. 27, 2023

(54) COMPOSITIONS FOR CORONAVIRUS INFECTION TREATMENT AND/OR PREVENTION

(71) Applicant: Fraunhofer-Gesellschaft zur Förderung der angewandten Forschung e.V., München (DE)

(72) Inventors: Philip GRIBBON, Hamburg (DE); Andrea ZALIANI, Hamburg (DE); Bernhard ELLINGER, Hamburg (DE); Gerd GEISSLINGER, Bad Soden (DE); Sandra CIESEK, Frankfurt am Main (DE); Jindrich CINATL, Offenbach (DE); Denisa BOJKOVA, Essen (DE)

(21) Appl. No.: 17/995,025

(22) PCT Filed: Apr. 1, 2021
(86) PCT No.: PCT/EP2021/058659

§ 371 Date: Sep. 29, 2022

(30) Foreign Application Priority Data
Apr. 2, 2020 (EP) 20167824.0
Nov. 18, 2020 (EP) 20208426.5

(51) Int. Cl. A61K 31/351; A61P 31/14; A61K 31/03; A61K 31/438; A61K 31/165; A61K 31/4545; A61K 45/06; A61K 9/12

(52) U.S. Cl. CPC A61K 31/351; A61P 31/14; A61K 31/03; A61K 31/438; A61K 31/165; A61K 31/4545; A61K 45/06; A61K 9/12

(57) ABSTRACT

A composition includes a compound for treating and/or preventing a coronavirus infection in a subject. The coronavirus is severe acute respiratory syndrome coronavirus (SARS-CoV)-2, SARS-CoV-1, or Middle East respiratory syndrome coronavirus (MERS-CoV). Methods include treating and/or preventing a coronavirus infection in a subject by administering the composition to the subject.

### PRIORITY AND CROSS REFERENCE TO RELATED APPLICATIONS

This application is the U.S. National Stage Application under 35 U.S.C. § 371 of International Application No. PCT/EP2021/058659, filed Apr. 1, 2021, designating the U.S. and published in English as WO 2021/198440 A1 on Oct. 7, 2021, which claims the benefit of European Application No. EP 20167824.0, filed Apr. 2, 2020 and European Application No. EP 20208426.5, filed Nov. 18, 2020. Any and all applications for which a foreign or a domestic priority is claimed is/are identified in the Application Data Sheet filed herewith and is/are hereby incorporated by reference in their entirety under 37 C.F.R. § 1.57.

### FIELD

The present invention relates to compositions for coronavirus infection treatment and/or prevention.

### BACKGROUND

The Coronaviridae typically cause mild respiratory diseases, but infections with β-coronavirus such as SARS-CoV-1, MERS and SARS-CoV-2 can cause acute respiratory diseases and high mortalities, especially in individuals with underlying health conditions. In the last 20 years, Coronaviridae have emerged in two severe outbreaks, 2002/2003 with SARS and 2012 with MERS. The recently described SARS-CoV-2-infection causes a disease named Covid-19. Multiple interventional clinical trials have been initiated in the search for effective pharmacological treatments against Covid-19 (Kupferschmidt & Cohen (2020), Science 367:1412, doi: 10.1126/science.367.6485.1412). Selection of single drug or combination treatments for Covid-19 trials has been based on previously described activities against SARS-CoV-1, Ebola, HIV and Malaria. Bioinformatics analyses have also proposed additional potential drugs based on the predicted interactome between viral proteins and host-cell pathways (Gordon et al. (2020), doi.org/10.1101/2020.03.22.002386v1). Overall, repurposing of existing drugs represents a pragmatic strategy for health authorities to meet patient needs in the absence of safe and effective vaccines, which is predicted to be the case for at least the first 12 months of the Covid-19 pandemic. Nevertheless, properly controlled clinical efficacy and safety studies will still need to be performed before the regulatory approval for any pharmacological treatment of Covid-19, even employing repurposed drugs.

### SUMMARY

The present invention relates to a composition comprising a compound selected from the compounds indicated in Table 1 and/or Table 2 for use in treating and/or preventing a coronavirus infection in a subject. The present invention further relates to methods and uses related thereto.

### BRIEF DESCRIPTION OF THE DRAWINGS

FIG. 1 shows dose response curves for exemplary compounds of Table 2; A: polidocanol, B: Amuvatinib (MP-470), C: AI-10-49, D: Tyrphostin (AG 879), E: Tanaproget, F: Chlormidazole; A) to D) are examples of inhibitors with a low IC50 (<1 μM), E), F) are examples of inhibitors with a medium IC50 (>1 μM).

FIG. 2 shows dose response curves for exemplary compounds in Examples 5 and 6; squares: % viability, circles: % infection after 48 h at an MOI of 0.1; A) Amuvatinib, B) Polidocanol, C) Luminespib, D) Salinomycin, E) Methotrexate, F) Flavopiridol, G) PF-04691502, and H) Pralatrexate.

FIG. 3 shows dose response curves for comparative compounds in Examples 5 and 6; squares: % viability, circles: % infection after 48 h at an MOI of 0.1; A) Pevonedistat, B) Nafamostat.

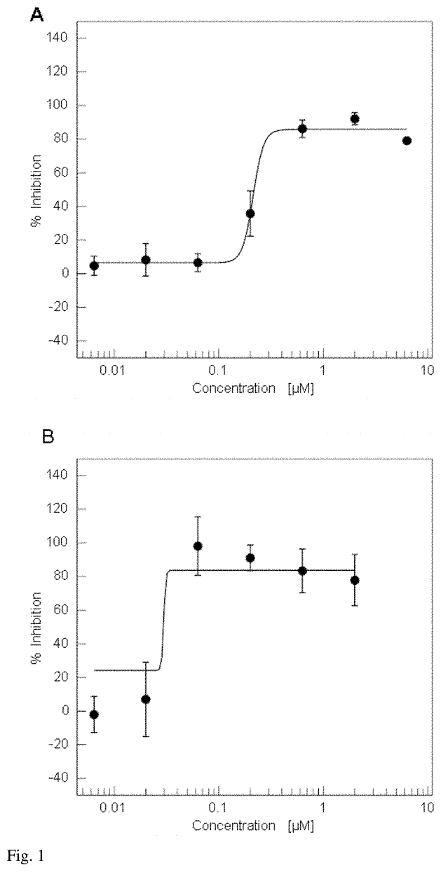

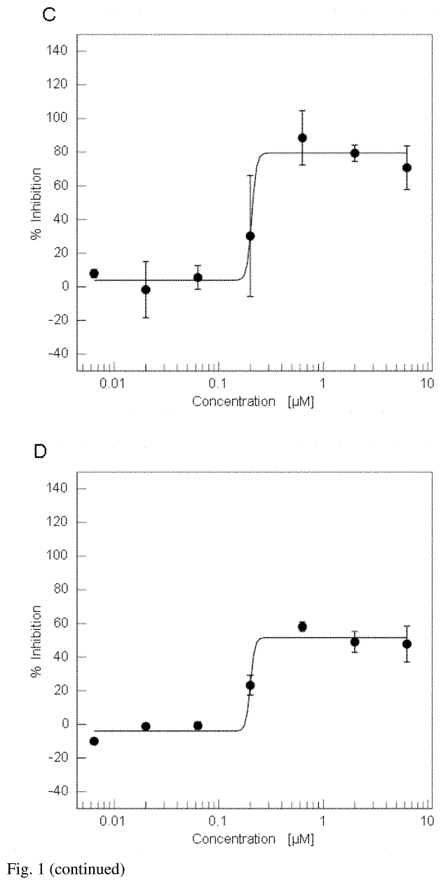

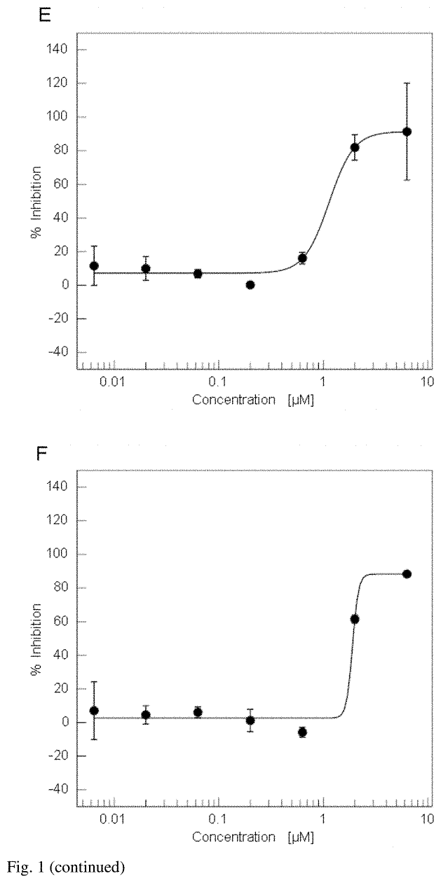

### DETAILED DESCRIPTION

There is a need in the art for improved means and methods for treating and/or preventing infection of coronavirus infection, avoiding the drawbacks of the prior art. This problem is solved by the means and methods disclosed herein with the features of the independent claims. Advantageous embodiments which might be realized in an isolated fashion or in any arbitrary combinations are listed in the dependent claims.

In accordance, the present invention relates to a composition comprising a compound selected from the compounds indicated in Table 1 for use in treating and/or preventing a coronavirus infection in a subject.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The term “composition”, as used herein, relates to a composition of matter comprising the compound as specified and optionally one or more acceptable carrier(s). Preferably, the composition is a pharmaceutic composition; thus, the composition, preferably, comprises the compound as specified as pharmaceutically active compound, and, preferably, the carrier is a pharmaceutically acceptable carrier. The pharmaceutically active compound can be formulated as, preferably pharmaceutically acceptable, salt. Preferred salts comprise acetate, methylester, sulfate, chloride, and the like. Further preferred salts are indicated elsewhere herein for the specific compounds. The composition comprises at least one compound as indicated; thus, the composition preferably comprises one compound as specified or comprises a multitude of compounds, the term “multitude”, preferably referring to at least two.

Preferably, the composition is a pharmaceutical composition, i.e., preferably, a medicament. The terms “medicament” and “pharmaceutical composition” are, in principle, known to the skilled person. As referred to herein, the term relate to any composition containing the compound as pharmaceutically active compound and one or more other components such as one or more pharmaceutically acceptable carrier(s). The pharmaceutically active compound can be present in liquid or dry, e.g. lyophilized, form. For example, the pharmaceutically active compound can be present together with glycerol and/or stabilizers (e.g., reducing agents, human serum albumin). The medicament is, typically, administered systemically or topically, preferably orally, by inhalation, or parenterally, e.g. by intravenous administration; however, preferably, subcutaneous or intramuscular administration may also be envisaged. However, depending on the nature of the formulation and the desired therapeutic application, the medicament may be administered by other routes as well. The pharmaceutically active compound is the active ingredient or drug of the medicament, and is preferably administered in conventional dosage forms prepared by combining the drug with standard pharmaceutical carriers according to conventional procedures. These procedures may involve mixing, granulating, and compression, or dissolving the ingredients as appropriate to the desired preparation. It will be appreciated that the form and character of the pharmaceutical acceptable carrier or diluent is dictated by the amount of active ingredient with which it is to be combined, the route of administration, and other well-known variables. The carrier(s) must be acceptable in the sense of being compatible with the other ingredients of the formulation and being not deleterious to the recipient thereof. The pharmaceutical carrier employed may include a solid, a gel, or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil, water, emulsions, various types of wetting agents, and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pa. The diluent(s) is/are selected so as not to affect the biological activity of the combination. Examples of such diluents are distilled water, physiological saline, Ringer's solutions, dextrose solution, and Hank's solution. In addition, the pharmaceutical composition or formulation may also include other carriers, adjuvants, or non-toxic, non-therapeutic, non-immunogenic stabilizers and the like. The medicament referred to herein is, preferably, administered at least once, e.g. as a bolus. However, the said medicament may be administered more than one time and, preferably, at least twice, e.g. permanently or periodically after defined time windows.

The term “compound”, as used herein, relates to a chemical compound selected from Table 1 or Table 2, preferably selected from the compounds shown in Table 2; Preferably, the compounds are the compounds identified by the CAS numbers indicated in Table 2. The compounds may, as the case may be, be used as free acid, free base, or a salt thereof; the skilled person preferably selects a pharmaceutically compatible acid, base, or salt of the compound as appropriate for the intended use and/or mode or administration. As indicated herein in the Examples, the compounds referred to herein are either approved for sale as pharmaceuticals or are actively developed for this purpose. Thus, the compounds and their structures are, in principle known to the skilled person and, preferably, a compound referred to herein is administered in the form, preparation, mode and/or dose as used for at least one known application of the compound. In view of the above, the compound, preferably is NSC319726. Preferably, the compound is Amuvatinib (MP-470). Preferably, the compound is Tyrphostin AG 879 (AG 879). Preferably, the compound is GSK2606414. Preferably, the compound is polidocanol. Preferably, the compound is AI-10-49. Preferably, the compound is VLX600. Preferably, the compound is Ethaverine, more preferably Ethaverine hydrochloride. Preferably, the compound is alvocidib. Preferably, the compound is Cycloheximide Preferably, the compound is Cetylpyridinium, more preferably Cetylpyridinium Chloride. Preferably, the compound is thioguanosine. Preferably, the compound is Dapivirine (TMC120). Preferably, the compound is LY2228820 (Ralimetinib). Preferably, the compound is Papaverine, more preferably Papaverine Hydrochloride. Preferably, the compound is Tanaproget. Preferably, the compound is Octenidine, more preferably Octenidine Dihydrochloride. Preferably, the compound is Almitrine, more preferably Almitrine dimesilate. Preferably, the compound is Sorafenib, more preferably Sorafenib Tosylate. Preferably, the compound is ZK-93423. Preferably, the compound is Thimerosal. Preferably, the compound is Regorafenib (BAY 73-4506). Preferably, the compound is Chlormidazole. Preferably, the compound is ravuconazole. Preferably, the compound is Methylene Blue. Preferably, the compound is Mibampator. Preferably, the compound is homoharringtonine. Preferably, the compound is hematoporphyrin. Preferably, the compound is LGK-974. Preferably, the compound is Posaconazole. Preferably, the compound is Ketoconazole. Preferably, the compound is Nelfinavir, more preferably Nelfinavir Mesylate. Preferably, the compound is JTE-013. Preferably, the compound is Bentamapimod. Preferably, the compound is RO5126766 (CH5126766). Preferably, the compound is Lonafarnib (SCH66336). Preferably, the compound is Apixaban. Preferably, the compound is Pexidartinib. Preferably, the compound is drotaverine. Preferably, the compound is LDE225 (NVP-LDE225. Preferably, the compound is Erismodegib. Preferably, the compound is PFK-015. Preferably, the compound is Avatrombopag. Preferably, the compound is 3′-fluorobenzylspiperone. Preferably, the compound is Etifoxine, more preferably Etifoxine hydrochloride. Preferably, the compound is Avasimibe (CI-1011). Preferably, the compound is Lopinavir (ABT-378). Preferably, the compound is DCPIB. Preferably, the compound is PH-797804. Preferably, the compound is flunarizine. Preferably, the compound is Loteprednol etabonate. Preferably, the compound is lidoflazine. Preferably, the compound is BP-897. Preferably, the compound is Cloconazole, more preferably Cloconazole HCl. Preferably, the compound is PF-670462. Preferably, the compound is Oxiconazole, more preferably Oxiconazole Nitrate. Preferably, the compound is AMG-9810. Preferably, the compound is Brexpiprazole. Preferably, the compound is talmapimod. Preferably, the compound is Adoprazine. Preferably, the compound is CC-223. Preferably, the compound is Harringtonine. Preferably, the compound is Barnidipine HCl. Preferably, the compound is vatalanib. Preferably, the compound is OSI-906 (Linsitinib). Preferably, the compound is Idarubicin, more preferably Idarubicin HCl. Preferably, the compound is Acrylflavine, more preferably Acriflavinium Chloride. Preferably, the compound is Basimglurant. Preferably, the compound is NNC-05-2090. Preferably, the compound is Proflavine, more preferably Proflavine Hemisulfate. Preferably, the compound is Evacetrapib (LY2484595). Preferably, the compound is LY335979 (Zosuquidar trihydrochloride). Preferably, the compound is prenylamine Preferably, the compound is Fosaprepitant, more preferably Fosaprepitant dimeglumine salt. Preferably, the compound is Mefloquine, more preferably Mefloquine Hydrochloride. Preferably, the compound is SB-657510. Preferably, the compound is P276-00. Preferably, the compound is PD-102807. Preferably, the compound is dexniguldipine. Preferably, the compound is ABC294640. Preferably, the compound is CGP-71683. In a preferred embodiment, the compound is aminopterin (CAS No. 54-62-6). In a further preferred embodiment, the compound is Methotrexate (CAS No. 59-05-2). In a further preferred embodiment, the compound is Flavopiridol (CAS No. 146426-40-6). In a further preferred embodiment, the compound is PF-04691502 (CAS No. 1013101-36-4). In a further preferred embodiment, the compound is Pralatrexate (CAS No. 146464-95-1).

The term “treating”, as used herein, refers to ameliorating or curing a disease or at least one symptom associated therewith. Thus, if there is amelioration or cure of the disease or at least a symptom associated therewith, the treatment shall be deemed to be effective. It will be understood that treating might not be effective in all subjects. However, according to the present invention it is envisaged that treatment will preferably be effective in at least a statistically significant portion of subjects to be treated. It is well known to the skilled artisan how to determine a statistically significant portion of subjects that can be effectively treated. Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the present invention allows that the finding of coronavirus infection will be correct for at least 60%, at least 70%, at least 80%, or at least 90% of the subjects of a given cohort or population.

The term “preventing” as used herein refers to avoiding the onset of the disease or at least one symptom associated therewith or to prevent the worsening of the disease or the said at least one symptom. The prevention as referred to herein can be typically achieved shortly after the compound is administered. If the administration stopped, however, the prevention may not persist for an unlimited time but may remain present for a certain preventive time window after application of the drug. Typically, a preventive time window in accordance with the present invention may be at least 1 day, at least 2 days, at least 3 days, at least 4 days, at least 5 days, at least 7 days, or at least two or four weeks. However, the preventive time window may also depend on the dosage of a peptide(s) as well as the mode of administration, the kind of formulation, and/or the number of administrations. For example, if a high dosage is applied, usually longer preventive time windows can be achieved. The same holds true if repeated doses are administered. It will be understood that prevention might not be effective in all subjects. However, according to the present invention it is envisaged that prevention preferably will be effective in at least a statistically significant portion of subjects. It is well known to the skilled artisan how to determine a statistically significant portion of subjects that can be effectively prevented. Whether a portion is statistically significant can be determined without further ado by the person skilled in the art using various well known statistic evaluation tools as discussed above. In view of the above, prevention, preferably, is vaccination against coronavirus infection.

Preferably, treating and/or preventing comprises inhibition of coronavirus replication. Also, preferably, treating and/or preventing comprises inhibition of cell lysis by said coronavirus. Further preferably, treating and/or preventing comprises inhibition of tissue destruction, preferably lung tissue destruction, by said coronavirus. As is understood by the skilled person, the term “inhibition” includes partial inhibition. Thus, in the case of a quantifiable event, inhibition preferably is a reduction of said event by at least 25%, more preferably at least 50%, still more preferably at least 75%, most preferably at least 85%. As will be understood by the skilled person, inhibition may also include complete inhibition, i.e. prevention of an event or process from occurring. Exemplary methods for determining inhibition by the compounds specified herein are provided herein in the Examples.

The term “coronavirus” is understood by the skilled person to relate to a group of enveloped viruses from the order Nidovirales, having a positive-sense single-stranded RNA genome with a size of approx. 25 to 35 kilobases. Preferably, the coronavirus is a beta-coronavirus, more preferably a severe acute respiratory syndrome coronavirus (SARS-CoV)-2, SARS-CoV-1, or Middle East respiratory syndrome coronavirus (MERS-CoV). Preferably the coronavirus is SARS-CoV-2. The terms “SARS-CoV-2” and “severe acute respiratory syndrome coronavirus 2” are understood by the skilled person. Preferably, the terms relate to the virus identified in Genbank entry NCBI:txid2697049. Symptoms and diseases caused by coronavirus infection and in particular SARS-CoV-2 infection are known to the skilled person. Also preferably the coronavirus is SARS-CoV-1 or Middle East respiratory syndrome coronavirus (MERS-CoV, NCBI:txid1335626).

The term “subject” as used herein refers to any kind of animal encompassing, e.g., mammals, birds, fish or reptiles. Typically, the animal, however, is a mammal such as a mammal used as a pet, including dogs, cats, horses, or rodents, laboratory animals, e.g., rats, mice or apes, or farming animals such as pigs, cows, goats, or sheep. More preferably, the mammal is a primate and, most preferably, a human. The subject according to the present invention shall preferably be known or suspected to suffer from coronavirus infection. Preferably, the subject is an animal known or suspected to be infectable by a coronavirus as specified, more preferably is a human. More preferably, in particular in case the compound is for use in treating a coronavirus infection, the subject has been diagnosed to suffer from coronavirus infection.

Advantageously, it was found in the work underlying the present invention that the compounds referred to herein are active in inhibiting replication of SARS-CoV-2 in a cell culture system, making the compounds candidates for treatment and prevention of coronavirus infections.

The definitions made above apply mutatis mutandis to the following. Additional definitions and explanations made further below also apply for all embodiments described in this specification mutatis mutandis.

The present invention further relates to a method of treating and/or preventing a coronavirus infection in a subject, comprising administering a compound of the resent invention to said subject, thereby treating and/or preventing said coronavirus infection in said subject.

The method of the present invention, preferably, is an in vivo method of treating a living subject. Moreover, it may comprise steps in addition to those explicitly mentioned above. For example, further steps may relate, e.g., to diagnosing coronavirus infection or determining a need for coronavirus prevention in a subject before step a), or providing additional treatment in step b). Moreover, one or more of said steps may be performed by automated equipment.

Treatment optionally administered in addition to administration of the compounds referred to herein preferably depends on severity of coronavirus infection and its symptoms. Thus, such additional treatment may comprise assistance with breathing, in particular administration of increased oxygen partial pressure, intubation, artificial respiration, treatment of sepsis, and the like. Further, the composition may be administered in combination with at least one pharmaceutical compound selected from the list consisting of zithromycin, Ribavirin, Interferons, Tocilizumab and Sarilumab (anti IL-6 monoclonal antibodies), Favirapir, Arbidol, Fingolimod, Siponimod, Colchicine, NO gas, and antibiotics, in particular erythromycin.

The instant invention also relates to a kit comprising a composition according to the present invention comprised in a housing.

The term “kit”, as used herein, refers to a collection of the aforementioned compounds, means or reagents which may or may not be packaged together. The components of the kit may be comprised by separate vials (i.e. as a kit of separate parts) or provided in a single vial. Moreover, it is to be understood that the kit of the present invention, preferably, is to be used for practicing the methods or uses referred to herein above. It is, preferably, envisaged that all components are provided in a ready-to-use manner for practicing the methods or uses referred to above. Further, the kit, preferably, contains instructions for carrying out said methods or uses. The instructions can be provided by a user's manual in paper or electronic form. In addition, the manual may comprise instructions for administration and/or dosage instructions using the kit of the present invention.

Preferably, the kit comprises a diluent and/or a means of administration. Appropriate diluents are described herein above; means of administration are all means suitable for administering the compound to a subject. Preferred means of administration are those known to the skilled person for the respective compound. The means of administration may include a delivery unit for the administration of the compound or composition and a storage unit for storing said compound or composition until administration. However, it is also contemplated that the means of the current invention may appear as separate devices in such an embodiment and are, preferably, packaged together in said kit. Preferred means for administration are those which can be applied without the particular knowledge of a specialized technician. In a preferred embodiment, the means for administration is a syringe, more preferably with a needle, comprising the compound or composition of the invention. In another preferred embodiment, the means for administration is an intravenous infusion (IV) equipment comprising the compound or composition. In still another preferred embodiment the means for administration is an inhaler comprising the compound of the present invention, wherein, more preferably, said compound is formulated for administration as an aerosol.

The present invention further relates to a use of a compound selected from the list consisting of the compounds indicated in Table 1 and/or Table 2 for the manufacture of a medicine for treating and/or preventing a coronavirus infection in a subject.

The present invention also relates to a method for identifying an inhibitor of coronavirus replication, comprising

a) contacting human colon carcinoma cells with a coronavirus in the presence of a candidate inhibitor of coronavirus replication,
b) determining virus replication in said human colon carcinoma cells a),
c) comparing virus replication determined in b) to a reference, and
d) based on the result of comparison step c), identifying an inhibitor of coronavirus replication.

The method of the present invention is an in vitro method. Moreover, the method may comprise steps in addition to those specifically mentioned, e.g. further steps may relate to further steps of determining and/or verifying viral growth in step b) and/or performing steps a) and b) in parallel in the absence to provide a reference. Preferably, some, more preferably all, steps are assisted or performed by automated equipment.

The term “inhibitor of coronavirus replication” is understood by the skilled person. Preferably, the term includes any and all compounds causing a reduced replication of a coronavirus in the method specified. Preferably, the inhibitor of coronavirus replication causes an inhibition of coronavirus replication, compared to an assay in the absence of the inhibitor, i.e. a reference, of at least 10%, more preferably at least 25%, most preferably at least 50%. In accordance, in case cell viability and/or cell replication are measured as parameters of coronavirus replication, the inhibitor of coronavirus replication preferably causes cell viability and/or cell replication, compared to an assay in the absence of the inhibitor, to be at least 75%, more preferably at least 90%, most preferably at least 95%. Also preferably, the inhibitor of coronavirus replication has an IC50 value of at most 50 μM, more preferably at most 25 μM, even more preferably at most 20 μM. In view of the above, the term “candidate inhibitor of coronavirus replication” relates to any compound suspected to be an inhibitor of coronavirus replication as specified above.

The term “human colon carcinoma cells” is understood by the skilled person to relate to any cultured cells, preferably cell line, derived from a human colon carcinoma. Preferably, the human colon carcinoma cells are CaCo-2 cells, which are commercially available from standard cell collections.

The term “virus replication”, as is understood by the skilled person, relates to any kind of production of progeny genomes by a virus, in particular a coronavirus. Preferably, the term includes production of progeny virus. Virus replication may, in principle, be determined by any means deemed appropriate by the skilled person, e.g. by determining viral polymerase activity, by determining the number or concentration of viral genomes, by determining the amount of viral capsid protein(s), and the like. Preferably, viral replication is determined by determining virus-induced cytotoxicity, preferably by determining cell count, cell viability, and/or cell proliferation. More preferably, determining virus replication comprises determining proliferation and/or viability of said human colon cancer cells by digital processing of pictures of unlabeled cells and/or quantification of stained nuclei. Methods for digital processing of pictures of unlabeled cells and/or quantification of stained nuclei are, in principle, known to the skilled person.

The term “reference” is understood by the skilled person. Preferably, the term relates to a value or range of value obtained in a control experiment. As indicated herein above, the method preferably comprises the additional step of incubating the human colon carcinoma cells, preferably under otherwise identical conditions, in the absence of inhibitor and candidate inhibitor (negative control), to determine the extent of virus replication in the absence of inhibitor. Thus, the reference may, e.g., preferably, be the number of viable cells at the end of the assay in the absence of inhibitor, the number of detectable cell nuclei at the end of the assay in the absence of inhibitor, or the number of viral genomes at the end of the assay in the absence of inhibitor. Also the reference may be a pre-determined value known from earlier experiments. Also preferably, the method comprises an additional step of incubating the human colon carcinoma cells, preferably under otherwise identical conditions, in the presence of an inhibitory concentration of a known inhibitor of inhibitor of coronavirus replication (positive control). Also preferably, a reference range or reference score may be determined, preferably from the values of the negative and positive control.

In view of the above, the following embodiments are particularly envisaged:

1. A composition comprising a compound selected from the compounds indicated in Table 2 and/or Table 1 for use in treating and/or preventing a coronavirus infection in a subject.
2. The composition for use of embodiment 1, wherein said compound is a compound selected from the list consisting of NSC319726, polidocanol, Amuvatinib (MP-470), Tyrphostin AG 879 (AG 879), GSK2606414, AI-10-49, VLX600, Ethaverine, preferably Ethaverine hydrochloride, alvocidib, Cycloheximide, Cetylpyridinium, preferably Cetylpyridinium Chloride, thioguanosine, Dapivirine (TMC120), LY2228820, Papaverine, preferably Papaverine Hydrochloride, Tanaproget, Octenidine, preferably Octenidine Dihydrochloride, Almitrine, preferably Almitrine dimesilate, Sorafenib, preferably Sorafenib Tosylate, ZK-93423, Thimerosal, Regorafenib (BAY 73-4506), Chlormidazole, ravuconazole, Methylene Blue, Mibampator, homoharringtonine, hematoporphyrin, LGK-974, Posaconazole, Ketoconazole, Nelfinavir, preferably Nelfinavir Mesylate, JTE-013, Bentamapimod, R05126766 (CH5126766), Lonafarnib (SCH66336), Apixaban, Pexidartinib, drotaverine, LDE225 (NVP-LDE225, Erismodegib, PFK-015, Avatrombopag, 3′-fluorobenzylspiperone, Etifoxine, preferably Etifoxine hydrochloride, Avasimibe (CI-1011), Lopinavir (ABT-378), DCPIB, PH-797804, flunarizine, Loteprednol etabonate, lidoflazine, BP-897, Cloconazole, preferably Cloconazole HCl, PF-670462, Oxiconazole, preferably Oxiconazole Nitrate, AMG-9810, Brexpiprazole, talmapimod, Adoprazine, CC-223, Harringtonine, Barnidipine HCl, vatalanib, OSI-906 (Linsitinib), Idarubicin, preferably Idarubicin HCl, Acrylflavine, preferably Acriflavinium Chloride, Basimglurant, NNC-05-2090, Proflavine, preferably Proflavine Hemisulfate, Evacetrapib (LY2484595), LY335979 (Zosuquidar trihydrochloride), prenylamine, Fosaprepitant, preferably Fosaprepitant dimeglumine salt, Mefloquine, preferably Mefloquine Hydrochloride, SB-657510, P276-00, PD-102807, dexniguldipine, ABC294640, and CGP-71683, or in a preferred embodiment selected from the list consisting of aminopterin, Methotrexate, Flavopiridol, PF-04691502, and Pralatrexate.
3. The composition for use of embodiment 1 or 2, wherein said coronavirus is a beta-coronavirus.
4. The composition for use of any one of embodiments 1 to 3, wherein said coronavirus is severe acute respiratory syndrome coronavirus (SARS-CoV)-2, SARS-CoV-1, or Middle East respiratory syndrome coronavirus (MERS-CoV).
5. The composition for use of any one of embodiments 1 to 4, wherein said coronavirus is SARS-CoV-2.
6. The composition for use of any one of embodiments 1 to 5, wherein said subject is a human.
7. The composition for use of any one of embodiments 1 to 6, wherein said treating and/or preventing comprises inhibition of coronavirus replication.
8. The composition for use of any one of embodiments 1 to 7, wherein said treating and/or preventing comprises inhibition of cell lysis by said coronavirus.
9. The composition for use of any one of embodiments 1 to 8, wherein said treating and/or preventing comprises inhibition of tissue destruction, preferably lung tissue destruction, by said coronavirus.
10. The composition for use of any one of embodiments 1 to 9, wherein said composition is a pharmaceutical composition.
11. The composition for use of any one of embodiments 1 to 10, wherein the inhibitor of coronavirus replication has an IC50 value of at most 50 μM, more preferably at most 25 μM, even more preferably at most 20 μM.
12. A method of treating and/or preventing a coronavirus infection in a subject, comprising administering a compound as specified in embodiment 1 or 2 to said subject, thereby treating and/or preventing said coronavirus infection in said subject.
13. Use of a compound selected from the list consisting of the compounds indicated in Table 1 and/or Table 2 for the manufacture of a medicine for treating and/or preventing a coronavirus infection in a subject.
14. A kit comprising a composition according to any one of embodiments 1 to 11 comprised in a housing.
15. The kit of embodiment 14, further comprising a diluent and/or a means of administration. 15.
16. The kit of embodiment 14 or 15, wherein the means for administration is an inhaler comprising said compound, preferably wherein said compound is formulated for administration as an aerosol.
17. A method for identifying an inhibitor of coronavirus replication, comprising
a) contacting human colon carcinoma cells with a coronavirus in the presence of a candidate inhibitor of coronavirus replication,
b) determining virus replication in said human colon carcinoma cells a),
c) comparing virus replication determined in b) to a reference, and
d) based on the result of comparison step c), identifying an inhibitor of coronavirus replication.
14. The method of embodiment 13, wherein said human colon carcinoma cells are CaCo-2 cells.
15. The method of embodiment 13 or 14, wherein said determining virus replication comprises determining proliferation and/or viability of said human colon cancer cells.
16. The method of any one of embodiments 13 to 5, wherein said determining virus replication comprises determining proliferation and/or viability of said human colon cancer cells by digital processing of pictures of unlabeled cells and/or quantification of stained nuclei.

All references cited in this specification are herewith incorporated by reference with respect to their entire disclosure content and the disclosure content specifically mentioned in this specification.

The following Examples shall merely illustrate the invention. They shall not be construed, whatsoever, to limit the scope of the invention.

### Example 1: Methods

Compound collection: A collection of 5632 compounds was assembled by an external partner (SPECS, Netherlands) in a manner aligned to the recommendations from the Broad Institute (Cambridge Mass. USA) (Corsello et al. (2017), Nature Medicine, 23:405-408, doi:10.1038/nm.4306). Specifically, compounds were controlled by LC/MS for purity and identity (minimum purity >95%). Compounds were stored in 100% DMSO at −20° C. The collection has 5632 unique compounds which includes 3400 compounds which have already reached clinical use across 600 indications, as well as 1582 pre-clinical compounds of varying degrees of validation. A curated database is available containing the compound, indication, primary target (where known) and mechanism of action, as well as analysis tools which can assist in mechanism of action determination and target elucidation (clue.io). These data have also been made available through the ChEMBL database.

Screening assay: To determine antiviral activity was performed based upon an adapted version of the protocol described in Bojkova et al. (2020), General Cell Biology & Physiology, doi: 10.21203/rs.3.rs-17218/v1. Surprisingly, and advantageously, it was found that Caco-2 cells can be used for determining coronavirus replication and that digital image processing and/or counting of stained nuclei can be used as a reliable surrogate marker of viral replication.

### Cell Culture

Human Caco-2 cells, derived from colon carcinoma, were obtained from the Deutsche Sammlung von Mikroorganismen and Zellkulturen (DSMZ; Braunschweig, Germany). Cells were grown at 37° C. in Minimal Essential Medium (MEM) supplemented with 10% fetal bovine serum (FBS) and containing 100 IU/ml penicillin and 100 μg/ml streptomycin. All culture reagents were purchased from Sigma.

Virus culture SARS-CoV-2 was isolated from samples of travellers returning from Wuhan (China) to Frankfurt (Germany) using human colon carcinoma cell line CaCo-2 as described previously (Bojkova et al. (2020), loc. cit.). SARS-CoV-2 stocks used in the experiments had undergone one passage on CaCo-2 cells and were stored at −80° C. Virus titers were determined as TCID50/ml in confluent cells in 96-well microtiter plates.

Cell viability assays for primary screening and dose response studies Compounds were added to confluent layers of CaCo-2 cells in MEM supplemented with 2% FBS in 96-well plates. For the primary screen (5632 compounds) final compound concentration was 10 μM (0.1% DMSO final) in singlicate. Dose response profiling of selected priority compounds used 3 replicates, each at 8 compound concentrations (maximum 20 μM, minimum=XnM half log dilution factor, 0.1% DMSO final). Following addition of compounds, cells were then immediately infected with SARS-CoV-2 at MOI 0.01. Control wells (+ virus and − virus) also contained DMSO at 0.1% DMSO final. After 48 hours, cells were fixed using 3% PFA in PBS, and the plates sealed and disinfected to inactivate SARS-CoV-2. Quantification of viral inhibition (based upon CaCo-2 cell viability relative to controls) was performed using high content imaging (Operetta CLS, PerkinElmer LAS GmbH, Germany). Firstly, in label free mode using digital phase contrast, with maximum contrast as a read out (Vicar et al. (2019), BMC Bioinformatics, doi: 10.1186/s12859-019-2880-8). Secondly, cell nuclei were stained (Hoechst). For both readouts, images were acquired using 10× objective with nine fields per well and analysed using the manufacturer's software (PerkinElmer, Columbus v.2.9.0.1546). For digital phase, the analysis sequence started with cell detection (method: c, common threshold: 0.05, area >100 μm2, splitting coefficient: 6.5, individual threshold: 0.05, contrast >0.05) and was followed by calculating morphology, intensity and position properties as well as cell confluence. Well level data were analysed using ActivityBase (IDBS, United Kingdom) and R (v.3.6.1). Test well results were normalized relative to the corresponding intra-plate control (no virus assigned as 100% inhibition, with virus assigned as 0% inhibition). Plate level statistical performance was assessed using the standard Z′ calculation.

### Compound Cytotoxity Against CaCo-2 Cells at 48 Hours

The cytotoxicity of the profiled compounds towards CaCo-2 cells at 48 hours was assessed. The CaCo-2 cells were plated in 384 imaging microplates and after reaching % confluency, compounds were added. After 48 hours, cytotoxicity was evaluated using both digital phase contrast readout as described above and ATP determination (Cell titre-glo, Promega Corp).

### Example 2: Results

Table 1 shows new compounds showing an at least 50% inhibition in the initial screening. Notably, in addition compounds such as camostat, thioguanine, and remdesivir were identified in the screening; these compounds were identified in other screens as well and were proposed for investigation in clinical trials (Gordon et al. (2020), doi.org/10.1101/2020.03.22.002386v1, Li & De Clercq (2020), Nat Rev Drug Discovery 19: 149), being proof that the screening method identifies suitable inhibitors of coronavirus replication.

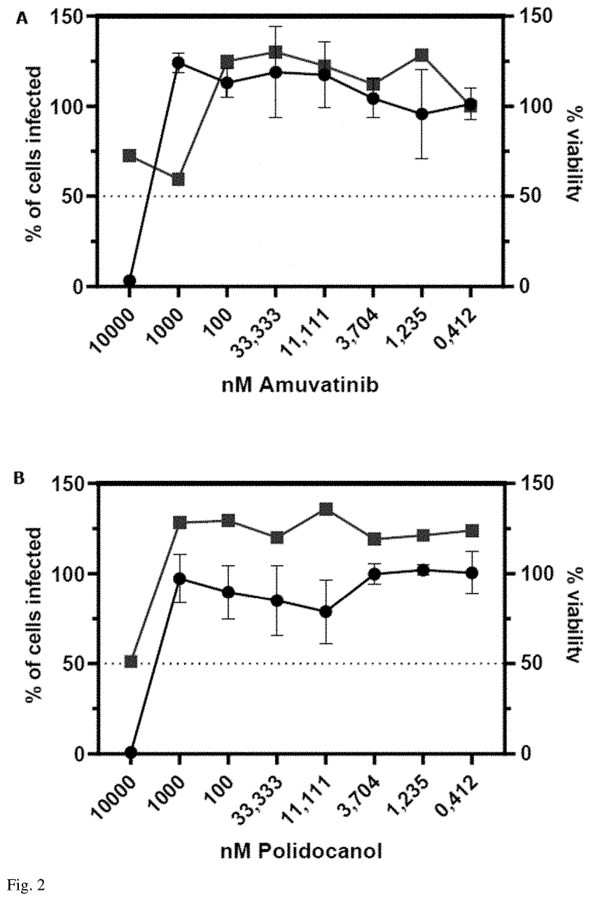

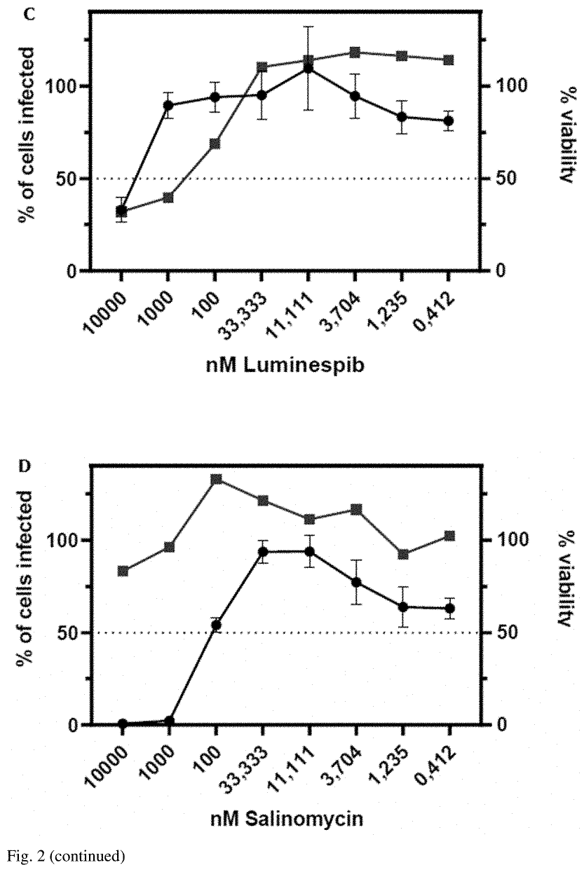

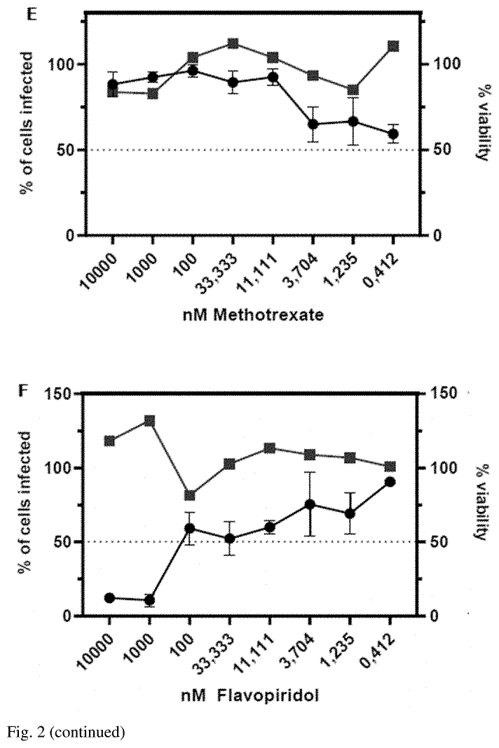

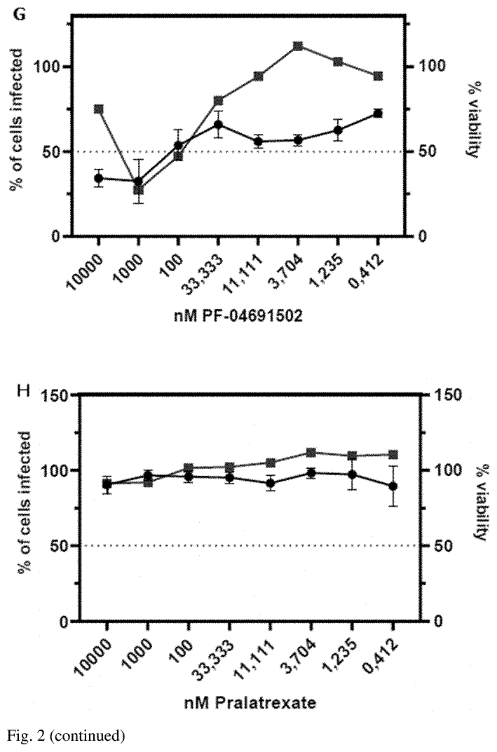

TABLE 1
Compounds causing at least 50% inhibition of SARS-CoV-2; % Inh: % Inhibition.

| Compound | % Inh. | | % Inh. |
|---|---|---|---|
| IPAG | 180.5 | BP-897 | 80.4 |
| homoharringtonine | 153.6 | 9-aminocamptothecin | 80.3 |
| SB-612111 | 146.6 | colforsin-daproate | 80.3 |
| verteporfin | 139.9 | DCPIB | 80.3 |
| CBiPES | 139.6 | Ku-0063794 | 80.1 |
| AC1NDSS5 | 131.0 | CLOSANTEL | 80.0 |
| AMG-PERK-44 | 127.9 | KX2-391 | 79.9 |
| PP-1 | 121.9 | EMD-53998 | 79.6 |
| PD-102807 | 121.8 | Dinaciclib (SCH727965) | 79.4 |
| REV-5901/L-655,238 | 113.5 | THIMEROSAL | 79.4 |
| VLX-600 | 113.3 | BIBX-1382 | 79.3 |
| Vistusertib (AZD2014) | 112.0 | SN-38 | 79.2 |
| PF-670462 | 111.6 | flunarizine | 79.1 |
| LY2228820 | 111.5 | U-0126 | 79.1 |
| Gemcitabine (elaidate) | 111.3 | nemorubicin | 78.9 |
| thioguanosine | 110.6 | SB-743921 | 78.9 |
| OSI-906 (Linsitinib) | 110.0 | AMODIAQUINE | 78.8 |
| | | DIHYDROCHLORIDE | |
| Onalespib (AT13387) | 109.9 | A-939572 | 78.8 |
| GSK2606414 | 109.9 | Sorafenib Tosylate | 78.7 |
| VLX600 | 109.8 | Pictilisib (GDC-0941) | 78.6 |
| ABAMECTIN (avermectin B1a) | 108.9 | BIBU-1361 | 78.5 |
| SC144 | 108.7 | Bentamapimod | 78.4 |
| Dapivirine (TMC120) | 108.5 | NI-57 | 78.3 |
| Thiostrepton | 108.2 | Fosaprepitant dimeglumine salt | 78.3 |
| PD-161570 | 108.1 | tecalcet | 78.1 |
| Masitinib (AB1010) | 107.9 | APIXABAN | 78.0 |
| GNF-7 | 107.6 | ESI-09 | 78.0 |
| Basimglurant | 107.5 | oxiperomide | 77.9 |
| JIB04 (NSC693627) | 106.4 | MK-5108 (VX-689) | 77.9 |
| Posaconazole | 106.3 | ZM-306416 | 77.9 |
| 4-mu-8C | 106.2 | trap-101 | 77.9 |
| JTE-013 | 105.9 | HKI-357 | 77.7 |
| WYE-687 | 105.8 | DDR1-IN-1 | 77.6 |
| ML-786 | 105.5 | Echinomycin | 77.5 |
| LE-135 | 105.5 | U-18666A | 77.4 |
| AZD8055 | 105.2 | Lomitapide | 77.4 |
| Fenticonazole nitrate | 105.2 | TC-S-7004 | 77.3 |
| LGK-974 | 105.0 | BNTX | 77.3 |
| OSI-027 | 104.8 | Bevantolol HCl | 77.1 |
| Luminespib | 104.5 | WAY-207024 | 77.1 |
| CC-223 | 104.4 | Octenidine Dihydrochloride | 77.0 |
| Lonafarnib (SCH66336) | 104.4 | CR8-(R) | 76.6 |
| Tyrphostin AG 879 (AG 879) | 104.2 | Trametinib | 76.6 |
| NSC319726 | 104.2 | lylamine | 76.4 |
| AI-10-49 | 103.8 | thonzonium bromide | 76.4 |
| Siramesine (hydrochloride) | 103.7 | BMS-599626 | 76.4 |
| PTC-209 | 103.6 | Alexidine dihydrochloride | 76.3 |
| TPCA-1 | 103.5 | Isoconazole nitrate (Travogen) | 75.9 |
| RS-17053 | 103.3 | TC-G-1005 | 75.5 |
| ravuconazole | 103.3 | AT-1015 | 75.4 |
| AS-1949490 | 103.1 | Torin 1 | 75.3 |
| RO5126766 (CH5126766) | 102.6 | tenidap | 75.2 |
| CGP-71683 | 102.5 | tetrindole | 75.2 |
| Loteprednol etabonate | 102.5 | ZK-93423 | 75.1 |
| 3′-fluorobenzylspiperone | 102.4 | LSN-2463359 | 74.9 |
| CHLORMIDAZOLE | 102.1 | pyrvinium | 74.7 |
| Apitolisib (GDC-0980, RG7422) | 101.9 | Retaspimycin (Hydrochloride) | 74.7 |
| LY3009120 | 101.6 | DACTINOMYCIN | 74.6 |
| Gemcitabine | 101.4 | budesonide | 74.4 |
| PFK-015 | 101.3 | LDN193189 (Hydrochloride) | 74.3 |
| N-(2-chlorophenyl)-2-({(2E)-2-[1-(2- | 101.3 | AG-1478 (Tyrphostin AG-1478) | 73.9 |
| pyridinyl)ethylidene]hydrazino}carb- | | | |
| othioyl)hydrazinecarbothioamide | | | |
| SB-657510 | 101.1 | FGIN-1-27 | 73.5 |
| ER-27319 maleate | 101.0 | NVP-TAE226 | 73.2 |
| TP-003 | 100.8 | TAK-285 | 73.0 |
| E 2012 | 100.8 | SNAP-5089 | 72.6 |
| TC-S-7006 | 100.7 | 2-chloroadenosine | 72.5 |
| ONC201 | 100.5 | piketoprofen | 72.4 |
| NVP-HSP990 | 100.4 | Rupatadine Fumarate | 72.1 |
| OSI-930 | 100.4 | AMN-082 | 71.9 |
| CETYLPYRIDINIUM CHLORIDE | 100.0 | NNC-05-2090 | 71.8 |
| AZ20 | 100.0 | KHK-IN-1 (hydrochloride) | 71.8 |
| WYE-125132 | 99.7 | NEFAZODONE | 71.7 |
| | | HYDROCHLORIDE | |
| Etifoxine (hydrochloride) | 99.7 | SALICYLIC ACID | 71.3 |
| AZ-628 | 99.6 | JX-401 | 71.2 |
| ACRIFLAVINIUM | 99.5 | casin | 71.2 |
| HYDROCHLORIDE | | | |
| remodelin | 99.4 | PPT | 71.1 |
| ML-277 | 99.3 | Apatinib mesylate | 70.9 |
| SIS3 | 99.3 | BIFONAZOLE | 70.8 |
| METHYLENE BLUE | 99.2 | Lanoconazole | 70.7 |
| WAY-600 | 99.1 | UCL-2077 | 70.7 |
| PD-158780 | 98.3 | BUTOCONAZOLE | 70.6 |
| BMS 794833 | 98.3 | DIGOXIN | 70.6 |
| TC-I-2014 | 97.8 | prolylleucylglycinamide | 70.6 |
| ACTB-1003 | 97.7 | SKF-96365 | 70.3 |
| BKM120 (NVP-BKM120) | 97.6 | RGB-286638 (free base) | 70.2 |
| dexniguldipine | 97.6 | Exatecan Mesylate | 70.1 |
| Triclocarban | 97.2 | DCC-2036 (Rebastinib) | 70.0 |
| PD153035 (Hydrochloride) | 97.2 | Entrectinib (RXDX-101) | 70.0 |
| Bimiralisib | 97.2 | propidium-iodide | 69.7 |
| CID-2011756 | 97.0 | ML-213 | 69.5 |
| Q-203 | 96.9 | Acriflavine hydrochloride | 69.4 |
| AZD3514 | 96.8 | Galeterone (TOK-001) | 69.0 |
| hematoporphyrin | 96.8 | BMS-833923 (XL139) | 68.8 |
| CC-115 | 96.6 | PF-915275 | 68.8 |
| SNX-2112 | 96.6 | Moxaverine | 68.7 |
| KN-62 | 96.5 | Marimastat | 68.7 |
| AZD3463 | 96.5 | SP-141 | 68.6 |
| CYCLOHEXIMIDE | 96.4 | toyocamycin | 68.4 |
| Tanaproget | 96.2 | SELAMECTIN | 68.2 |
| Pexidartinib | 96.1 | Icotinib | 68.1 |
| algestone-acetophenide | 96.0 | lazabemide | 67.9 |
| Avatrombopag | 95.9 | PF-2545920 | 67.8 |
| lidoflazine | 95.9 | RESERPINE | 67.8 |
| QNZ (EVP4593) | 95.8 | SR-33805 oxalate | 67.7 |
| PROFLAVINE HEMISULFATE | 95.8 | NPS-2143 (SB262470) | 67.3 |
| PLX-647 | 95.7 | 1-(4-fluorophenyl)-4-(3,4,5- | 67.3 |
| | | trimethoxybenzyl)piperazine | |
| OF-1 | 95.7 | AZD5438 | 67.2 |
| BETAINE HYDROCHLORIDE | 95.7 | HETACILLIN POTASSIUM | 67.1 |
| VS-5584 | 95.5 | phortress | 67.1 |
| Tocofersolan | 95.2 | Strophantidin | 67.1 |
| GSK2656157 | 95.0 | MEK1-2-inhibitor | 66.9 |
| norfluoxetine | 95.0 | Digoxigenin | 66.8 |
| EC-144 | 95.0 | Chlorophyllin (sodium copper | 66.7 |
| | | salt) | |
| Evacetrapib (LY2484595) | 94.9 | GBR-13069 | 66.7 |
| JNJ-42165279 | 94.9 | gossypol | 66.3 |
| CGP-52411 | 94.5 | nocodazole | 66.1 |
| P276-00 | 94.4 | EO-1428 | 66.1 |
| OXYCLOZANIDE | 94.4 | Bardoxolone | 66.0 |
| Carnosine | 94.3 | pyrrolidine-1-carbonitrile | 65.9 |
| sulforaphane | 94.2 | DEQUALINIUM CHLORIDE | 65.6 |
| TC-E-5006 | 94.1 | sorbic-acid | 65.5 |
| SX-011 | 94.1 | PDP-EA | 65.3 |
| 1-naphthyl-PP1 | 94.1 | TC-Mps1-12 | 65.3 |
| PIMOZIDE | 94.0 | JK-184 | 65.2 |
| JNJ 1661010 | 93.8 | mivobulin | 65.0 |
| AMG-9810 | 93.7 | N-oxydiethylenebenzothiazole- | 64.6 |
| | | 2-sulfenamide | |
| Harringtonine | 93.6 | SB705498 | 64.6 |
| LDE225 (NVP-LDE225, | 93.5 | levobunolol | 64.5 |
| Erismodegib) | | | |
| MEFLOQUINE HYDROCHLORIDE | 93.3 | Miglustat (n- | 64.4 |
| | | Butyldeoxynojirimycin) | |
| Almitrine dimesilate | 93.2 | Dolastin-10 | 64.3 |
| RAF265 (CHIR-265) | 93.2 | Acenocoumarin | 64.2 |
| prenylamine | 93.2 | GSK9027 | 64.1 |
| JHW-007 | 93.1 | AMINACRINE | 64.1 |
| drotaverine | 93.1 | pirenperone | 64.1 |
| LY335979 (Zosuquidar | 93.0 | RHC-80267 | 63.9 |
| trihydrochloride) | | | |
| niguldipine-(S)-(+) | 93.0 | LANATOSIDE C | 63.7 |
| HEXACHLOROPHENE | 92.9 | NAB-2 | 63.3 |
| Abafungin | 92.9 | AC-55649 | 63.1 |
| Cloconazole HCl | 92.8 | 4-PPBP | 63.1 |
| Isavuconazole | 92.8 | ABT-737 | 63.1 |
| NTNCB | 92.7 | hemin | 62.9 |
| LY3023414 | 92.7 | Ansamitocin P-3 | 62.6 |
| ML-365 | 92.7 | cinnamaldehyde | 62.3 |
| TAS-103 (dihydrochloride) | 92.6 | ASP3026 | 62.2 |
| GSK1059615 | 92.4 | AFALANINE | 62.1 |
| talmapimod | 92.3 | ACRISORCIN | 61.7 |
| spautin-1 | 92.1 | Cobimetinib | 61.6 |
| ML-9 | 91.9 | MPI-0479605 | 61.2 |
| Ispinesib (SB-715992) | 91.9 | Fomocain | 61.1 |
| Dactolisib (BEZ235, NVP-BEZ235) | 91.8 | Brequinar sodium salt hydrate | 61.0 |
| PFI-4 | 91.7 | NP118809 | 60.8 |
| TAK-960 | 91.6 | YM-244769 | 60.4 |
| Regorafenib (BAY 73-4506) | 91.5 | A-784168 | 60.3 |
| SNAP-94847 | 91.4 | CO-101244 | 60.2 |
| GW-3965 | 91.4 | Hycanthone | 60.2 |
| Halofantrine | 91.4 | acetylsalicylsalicylic-acid | 60.2 |
| Avasimibe (CI-1011) | 91.4 | Afatinib (dimaleate) | 60.2 |
| PF-04929113 (SNX-5422) | 91.3 | BU-239 | 60.1 |
| KHS-101 | 91.2 | NM107, 2′-C-methylcytidine | 60.1 |
| MBX-2982 | 91.1 | GW-583340 | 60.1 |
| DAA-1106 | 91.0 | digitoxigenin | 60.0 |
| Revaprazan Hydrochloride | 90.9 | PREDNISOLONE ACETATE | 60.0 |
| AEE788 | 90.9 | Ceritinib | 60.0 |
| ZSTK474 | 90.9 | CTEP (RO4956371) | 59.7 |
| Triptolide | 90.7 | SGC-CBP30 | 59.2 |
| NAFTOPIDIL | 90.6 | QUININE ETHYL | 59.1 |
| | | CARBONATE | |
| sertindole | 90.3 | antalarmin | 59.0 |
| SULCONAZOLE NITRATE | 90.3 | LOPERAMIDE | 58.9 |
| | | HYDROCHLORIDE | |
| QUINACRINE HYDROCHLORIDE | 90.3 | FR-180204 | 58.8 |
| Salinomycin (Procoxacin) | 90.2 | 7-methoxytacrine | 58.7 |
| Mibampator | 90.1 | PF-03882845 | 58.6 |
| tafenoquine | 90.0 | CYT997 | 58.6 |
| 4-IBP | 89.9 | zamifenacin | 58.3 |
| SMI-4a | 89.9 | SULFACARBAMIDE | 58.1 |
| WYE-354 | 89.9 | NNC-55-0396 | 58.1 |
| INH1 | 89.8 | PRAZOSIN | 58.0 |
| | | HYDROCHLORIDE | |
| Amuvatinib (MP-470) | 89.4 | Antagonist-G | 58.0 |
| LY266097 | 89.3 | 2-chloro-N6- | 57.7 |
| | | cyclopentyladenosine | |
| SCR-1481B1 | 89.3 | SB-216641 | 57.6 |
| LY2857785 | 89.1 | rose-bengal-lactone | 57.6 |
| AMG-548 | 89.0 | cabozantinib | 57.5 |
| Sertaconazole nitrate | 88.9 | PU-H71 | 57.4 |
| Actinomycin D | 88.9 | Palonosetron HCl | 57.4 |
| Nelfinavir Mesylate | 88.6 | R306465 | 57.3 |
| RG7112 (RO5045337) | 88.5 | SR-59230A | 57.3 |
| KN-93 | 88.4 | etanidazole | 57.3 |
| GSK2636771 | 88.1 | SODIUM OXYBATE | 57.3 |
| anisomycin | 88.1 | 1,3-dipropyl-8-phenylxanthine | 57.2 |
| alvocidib | 87.8 | Benzyldimethylhexadecylammonium | 57.0 |
| | | chloride hydrate | |
| ML314 | 87.7 | spiperone | 56.9 |
| CETRIMONIUM BROMIDE | 87.7 | Lopinavir (ABT-378) | 56.6 |
| PAPAVERINE HYDROCHLORIDE | 87.7 | SB1317 | 56.5 |
| PIK-75 | 87.6 | hydroxypropyl-beta-cyclodextrin | 56.5 |
| CH55 | 87.5 | KG-5 | 56.3 |
| BUMETANIDE | 87.5 | lofemizole | 56.3 |
| NITROFURAZONE | 87.3 | HYDRASTININE | 56.3 |
| | | HYDROCHLORIDE | |
| ECONAZOLE NITRATE | 87.3 | Azeliragon | 56.0 |
| 4-CMTB | 87.1 | I-BET151 (GSK1210151A) | 56.0 |
| KETOCONAZOLE | 87.1 | Dazoxiben | 56.0 |
| CP-471474 | 86.9 | adipic-acid | 55.9 |
| Ercalcitriol | 86.9 | CHLORPROTHIXENE | 55.6 |
| | | HYDROCHLORIDE | |
| iobenguane | 86.9 | diphenyleneiodonium | 55.5 |
| CCT128930 | 86.7 | PERHEXILINE MALEATE | 55.4 |
| Idarubicin HCl | 86.6 | TG003 | 55.4 |
| fendiline | 86.5 | chlorotrianisene | 55.3 |
| CHLORHEXIDINE | 86.4 | PCA-4248 | 55.3 |
| DIHYDROCHLORIDE | | | |
| BCTC | 86.4 | co-102862 | 55.2 |
| CGP-53353 | 86.1 | SD-208 | 55.1 |
| Endoxifen HCl | 86.1 | icilin | 55.1 |
| Skepinone-L | 86.1 | GP2a | 54.9 |
| PARICALCITOL | 86.0 | METERGOLINE | 54.6 |
| Temoporfin | 85.9 | PENTAMIDINE | 54.6 |
| | | ISETHIONATE | |
| vatalanib | 85.9 | metipranolol | 54.6 |
| PENFLURIDOL | 85.9 | A66 | 54.4 |
| polidocanol | 85.8 | CGP-60474 | 54.4 |
| AR-12 | 85.7 | CP 101606 | 54.3 |
| Teijin-compound-1 | 85.7 | Lasofoxifene (Tartrate) | 54.3 |
| Dacomitinib (PF299804, PF- | 85.5 | thiocolchicoside | 54.2 |
| 00299804) | | | |
| Ciclesonide | 85.4 | dipivefrine | 54.0 |
| PF-750 | 85.2 | L-733060 | 53.9 |
| ETHAVERINE HYDROCHLORIDE | 85.0 | ACT-462206 | 53.8 |
| INK 128 (MLN0128) | 84.9 | GR-127935 | 53.7 |
| Rociletinib (CO-1686, AVL-301) | 84.9 | Cephalothin | 53.7 |
| Brexpiprazole | 84.8 | Cevipabulin | 53.6 |
| Voxtalisib | 84.3 | trans-4-Methoxycinnamic-acid | 53.6 |
| SANT-2 | 84.1 | A-674563 | 53.3 |
| PSN-375963 | 83.9 | TC1 | 53.2 |
| Taltobulin (trifluoroacetate) | 83.8 | Pimasertib (AS-703026) | 53.2 |
| GDC-0349 | 83.8 | Ospemifene | 52.9 |
| Bamidipine HCl | 83.5 | ARV-825 | 52.8 |
| SC-9 | 83.4 | Oprozomib (ONX 0912) | 52.7 |
| Pelitinib (EKB-569) | 83.4 | Dasatinib | 52.7 |
| OXICONAZOLE NITRATE | 83.3 | TCS-OX2-29 | 52.7 |
| DR-4485 | 82.8 | Vigabatrin | 52.5 |
| iCRT-14 | 82.5 | mibefradil | 52.4 |
| AZD7545 | 82.4 | Nizofenone fumarate | 52.4 |
| AR-A014418 | 82.3 | THEOBROMINE | 52.4 |
| Wnt-C59 (C59) | 82.3 | BMS-191011 | 52.3 |
| mubritinib | 82.2 | HA-966-(S)-(−) | 52.3 |
| Rilpivirine | 82.1 | NU-2058 | 51.9 |
| RS-102895 | 82.1 | PD-198306 | 51.6 |
| camptothecin | 81.8 | AMG-073 HCl (Cinacalcet | 51.6 |
| | | hydrochloride) | |
| R547 | 81.8 | PD168393 | 51.5 |
| CLOTRIMAZOLE | 81.7 | IPRIFLAVONE | 51.4 |
| TC-F-2 | 81.7 | Vandetanib (ZD6474) | 51.2 |
| PH-797804 | 81.5 | FELODIPINE | 51.2 |
| SNS-314 methanesulfonate | 81.4 | verubulin | 50.9 |
| ABC294640 | 81.4 | FK-866 | 50.9 |
| Adoprazine | 81.4 | oxfenicine | 50.7 |
| PYRONARIDINE | 80.6 | TIOCONAZOLE | 50.6 |
| TETRAPHOSPHATE | | | |
| isoflupredone-acetate | 80.6 | BETAMETHASONE | 50.6 |
| LY-404187 | 80.4 | BHQ | 50.6 |
| | | merbarone | 50.4 |
| Other preferred compounds | | ML-3403 | 50.3 |
| (e.g. FIG. 2) | | | |
| Aminopterin | | Afloqualone | 50.1 |
| Methotrexate | | niceritrol | 50.1 |
| Flavopiridol | | isotiquimide | 50.0 |
| PF-04691502 | | anthranilic-acid | 50.0 |
| Pralatrexate | | | |

From the dose response experiments, IC50 and pIC50 values were calculated for the compounds tested. Compounds with an pIC50 value of at least 4 are shown in Table 2.

TABLE 2
Compounds with an pIC50 of at least 4.

| | IC50 | | |
|---|---|---|---|
| Compound | (μM) | pIC50 | CAS NO: |
| LY2228820 | 0.87 | 6.06 | 862507-23-1 |
| Amuvatinib (MP-470) | 0.03 | 7.52 | 850879-09-3 |
| Tyrphostin AG 879 (AG 879) | 0.2 | 6.70 | 148741-30-4 |
| GSK2606414 | 0.2 | 6.70 | 1337531-89-1 |
| polidocanol | 0.21 | 6.68 | 9006-65-9 |
| AI-10-49 | 0.21 | 6.68 | 1256094-72-0 |
| VLX600 | 0.23 | 6.64 | 327031-55-0 |
| ETHAVERINE | 0.54 | 6.27 | 985-13-7 |
| HYDROCHLORIDE | | | |
| alvocidib | 0.56 | 6.25 | 146426-40-6 |
| Cycloheximide | 0.57 | 6.24 | 66-81-9 |
| CETYLPYRIDINIUM CHLORIDE | 0.62 | 6.21 | 123-03-5 |
| thioguanosine | 0.69 | 6.16 | 85-31-4 |
| Dapivirine (TMC120) | 0.71 | 6.15 | 244767-67-7 |
| NSC319726 | 0.01 | 8.00 | 71555-25-4 |
| PAPAVERINE | 0.95 | 6.02 | 61-25-6 |
| HYDROCHLORIDE | | | |
| Tanaproget | 1.13 | 5.95 | 304853-42-7 |
| Octenidine Dihydrochloride | 1.14 | 5.94 | 64690-21-7 |
| Almitrine dimesilate | 1.21 | 5.92 | 29608-49-9 |
| Sorafenib Tosylate | 1.21 | 5.92 | 284461-73-0 |
| ZK-93423 | 1.28 | 5.89 | 1216574-52-5 |
| THIMEROSAL | 1.77 | 5.75 | 54-64-8 |
| Regorafenib (BAY 73-4506) | 1.79 | 5.75 | 755037-03-7 |
| CHLORMIDAZOLE | 1.9 | 5.72 | 3689-76-7 |
| ravuconazole | 2 | 5.70 | 182760-06-1 |
| METHYLENE BLUE | 2.06 | 5.69 | 61-73-4 |
| Mibampator | 2.23 | 5.65 | 375345-95-2 |
| homoharringtonine | 2.25 | 5.65 | 26833-87-4 |
| hematoporphyrin | 2.31 | 5.64 | 14459-29-1 |
| LGK-974 | 2.33 | 5.63 | 1243244-14-5 |
| Posaconazole | 2.37 | 5.63 | 171228-49-2 |
| KETOCONAZOLE | 2.41 | 5.62 | 65277-42-1 |
| Nelfinavir Mesylate | 2.65 | 5.58 | 159989-65-8 |
| JTE-013 | 3.66 | 5.44 | 383150-41-2 |
| Bentamapimod | 5.12 | 5.29 | 848344-36-5 |
| RO5126766 (CH5126766) | 5.36 | 5.27 | 946128-88-7 |
| Lonafarnib (SCH66336) | 5.66 | 5.25 | 193275-84-2 |
| APIXABAN | 5.88 | 5.23 | 503612-47-3 |
| Pexidartinib | 5.92 | 5.23 | 1029044-16-3 |
| drotaverine | 6.18 | 5.21 | 985-12-6 |
| LDE225 (NVP-LDE225, | 7.03 | 5.15 | 956697-53-3 |
| Erismodegib) | | | |
| PFK-015 | 7.03 | 5.15 | 4382-63-2 |
| Avatrombopag | 7.17 | 5.14 | 570406-98-3 |
| 3′-fluorobenzylspiperone | 7.59 | 5.12 | 1135278-61-3 |
| Etifoxine (hydrochloride) | 9.29 | 5.03 | 21715-46-8 |
| Avasimibe (CI-1011) | 10.87 | 4.96 | 166518-60-1 |
| Lopinavir (ABT-378) | 20.3 | 4.69 | 369372-47-4 |
| DCPIB | 20.51 | 4.69 | 82749-70-0 |
| PH-797804 | 20.65 | 4.69 | 586379-66-0 |
| flunarizine | 20.69 | 4.68 | 52468-60-7 |
| Loteprednol etabonate | 21.29 | 4.67 | 82034-46-6 |
| lidoflazine | 21.34 | 4.67 | 3416-26-0 |
| BP-897 | 21.5 | 4.67 | 314776-92-6 |
| Cloconazole HCl | 21.53 | 4.67 | 77175-51-0 |
| PF-670462 | 21.96 | 4.66 | 950912-80-8 |
| OXICONAZOLE NITRATE | 22.33 | 4.65 | 64211-46-7 |
| AMG-9810 | 22.78 | 4.64 | 545395-94-6 |
| Brexpiprazole | 22.86 | 4.64 | 913611-97-9 |
| talmapimod | 22.93 | 4.64 | 309913-83-5 |
| Adoprazine | 23.38 | 4.63 | 222551-17-9 |
| CC-223 | 23.55 | 4.63 | 1228013-30-6 |
| Harringtonine | 23.65 | 4.63 | 26833-85-2 |
| Barnidipine HCl | 24.32 | 4.61 | 104757-53-1 |
| vatalanib | 24.45 | 4.61 | 212141-54-3 |
| OSI-906 (Linsitinib) | 24.92 | 4.60 | 867160-71-2 |
| Idarubicin HCl | 24.96 | 4.60 | 57852-57-0 |
| ACRIFLAVINIUM | 25.11 | 4.60 | 8063-24-9 |
| HYDROCHLORIDE | | | |
| Basimglurant | 25.15 | 4.60 | 1034442-21-1 |
| NNC-05-2090 | 25.21 | 4.60 | 184845-18-9 |
| PROFLAVINE HEMISULFATE | 26.69 | 4.57 | 1811-28-5 |
| Evacetrapib (LY2484595) | 26.71 | 4.57 | 1186486-62-3 |
| LY335979 (Zosuquidar | 26.82 | 4.57 | 167354-41-8 |
| trihydrochloride) | | | |
| prenylamine | 27.17 | 4.57 | 390-64-7 |
| Fosaprepitant dimeglumine salt | 28.68 | 4.54 | 265121-04-8 |
| MEFLOQUINE | 28.88 | 4.54 | 51773-92-3 |
| HYDROCHLORIDE | | | |
| SB-657510 | 29.51 | 4.53 | 474960-44-6 |
| P276-00 | 30.77 | 4.51 | 920113-03-7 |
| PD-102807 | 32.95 | 4.48 | 23062-91-1 |
| dexniguldipine | 35.78 | 4.45 | 120054-86-6 |
| ABC294640 | 38.17 | 4.42 | 915385-81-8 |
| CGP-71683 | 71.55 | 4.15 | 192322-50-2 |

### Example 3: Computer-Aided Drug Design

The EXSCALATE platform, the most powerful computing resources currently based in Europe to empower smart in-silico drug design (co-funded by the H2020-FET-HPC ANTAREX project), was exploited to perform High Performance Computing (HPC) simulations, with the final aim to select molecules active against the SARS-CoV-2 virus. The crystal structures of the main functional units of SARS-CoV-2 proteome were obtained from the Protein Data Bank; Table 3 reports the list of the proteins analysed, with the corresponding PDB codes. Homology models of the proteins for which the crystal structure is not available were generated and used. In particular the SARS-CoV-2 proteins most interesting as target to identify antiviral drugs, were selected.

TABLE 3
SARS-CoV-2 proteins analyzed and their PDB accession numbers.

| Proteins | PDB code |
|---|---|
| 3CL protease | 6LU7 |
| N-protein | 6VYO |
| NSP3 | 6W02 |
| NSP6 | De novo model |
| NSP9 | 6W4B |
| NSP12 | 7BV2 |
| NSP13 | Homology Model |
| NSP14 | Homology Model |
| NSP15 | 6W01 |
| NSP16 | 6W4H |
| PL protease | 6W9C |
| Spike-ACE2 | 6M0J |

Molecular dynamics simulations on the SARS-CoV-2 proteins were performed to explore the conformational space of their active sites, and select several protein conformations particularly suitable for docking simulations. MD simulations were carried out on the protein structures, prepared ad hoc in order to optimize the 3D structure from a chemical and conformational point of view. The structure was firstly subjected to a cycle of energy minimization by steepest descent methods to eliminate all initial steric clashes and obtain a pre-equilibrated model to start from. Then a 100 ps restrained MD simulation (typically 1000 KJ/mole force constant) was performed on the solvent atoms to equilibrate water molecules keeping the solute restrained. Finally, a production run was performed to generate a 1 microsecond trajectory with a total of 20.000 collected. Post HPC-run analysis of the results was performed.

High Performance Computing (HPC) simulation was conducted to virtual screen against the SARS-CoV-2 proteins, the Safe in Man (SIM) library, containing commercialized and under development drugs, already proved safe in man (>10,000 drugs), and the Fraunhofer's BROAD Repurposing Library, containing 5400 marketed drugs and clinical stage compounds and molecules with known mode of action. Duplicates between the libraries were removed before ligand preparation, where all compounds were converted to 3D and prepared with Schrödinger's LigPrep tool. This process generated multiple states for stereoisomers, tautomers, ring conformations (1 stable ring conformer by default) and protonation states. In particular, another Schrödinger package, Epik, was used to assign tautomers and protonation states that would be dominant at a selected pH range (pH=7±1). Ambiguous chiral centers were enumerated, allowing a maximum of 32 isomers to be produced from each input structure. Then, an energy minimization was performed with the OPLS3 force.

The simulation was performed using LiGen™ (Ligand Generator), the de novo structure based virtual screening software, designed and developed to run on HPC architectures, which represent the most relevant tool of the EXSCALATE platform. LiGen™ is formed by a set of tools that can be combined in a user-defined manner to generate project centric workflows. In particular, LiGenDock is a docking module using LiGenScore to compute the scoring function and the LiGenPass and LiGenPocket modules to obtain the 3D structure of the binding site. Both the docking algorithm implemented in LiGen™, the pharmacophoric docking (LiGenPh4) and the geometrical docking (LiGenGeodock), as well as the different scoring functions calculated, were used in this study to explore different protocols of Virtual Screening (VS) and select the best one in terms of performance.

Docking settings were validated through either redocking (where possible) or docking of known ligands. When tautomers were involved, only the one, with the best docking score, was chosen. The docking score values that predict the binding affinity of the molecules in the protein binding site, are reported (the higher, the better).

Virtual screening was performed on the main SARS-CoV-2 proteins to evaluate the potential poly-pharmacological effect of the compounds on the COVID-19 virus. A total score, corresponding to the sum of the docking scores obtained for each protein, was also calculated for each molecule and used to prioritize the most interesting molecules. Table 4 below reports the results obtained for the best scored molecules, selected for further validation of the antiviral activity in in vitro experiments.

### Example 4: In Vitro Screening of Activity Against SARS-CoV-2

The compounds selected as active by the CADD analysis (Example 3) were tested in a SARS-CoV 2 VeroE6-EGFP HTS antiviral Assay. This assay is based on the monitoring of cytopathic effects induced by viral infection of VeroE6 cells constitutively expressing an enhanced green fluorescent protein (EGFP). In details, the VeroE6-EGFP reporter cell line was received from JNJ whereas the SARS-CoV-2 used for infections is a Belgian strain (BetaCov/Belgium/GHB-03021/2020) present in the KU Leuven laboratories.

The test compounds were dissolved at 10 mM in dimethylsulphoxide (DMSO) and then diluted in cell culture medium to the required final concentrations. DMSO final concentration was below 0.5%. Then, compounds were mixed with 0.01 MOI of the virus 8000 VeroE6-EGFP cells/well in 384-well plates. Cells infected by Sars-CoV 2 and treated with Remdesivir 20 uM were used as positive control (inhibition 100%) whereas cells infected by Sars-CoV 2 without Remdesivir as negative control (0% inhibition).

After incubation at 37° C. for 5 days the EGFP signal of each well was recorded using an argon laser-scanning microscope. Standard whole-well fluorescence plate readout was performed (self-optimizing protocol, —6 min per 384-well plate, 4 reads/well) and the fluorescence total intensity was measured for both test compounds and control wells.

High-content imaging readout was also performed using a 5× objective, allowing to capture almost the entire well of a 384-well plate at once (auto-focus on each well, no binning, 1 channel). Two values were obtained from high content imaging:
% Confluence: indicates the increase in confluence of VEROE6-EGFP cells in the test compound wells compared to the control wells. This parameter is based on the quantification of the total surface of the field that gives a green fluorescent signal, due to the presence of EGPF-positive cells in SARS-CoV-2 infected wells treated with test compounds compared to untreated control wells: higher values mean that there are a large number of cells on the microtiter plate bottom surface, small values mean that most of the fluorescence is lost (i.e. the cells died). In the table attached, we reported the Maximum confluence reached (%) after test compound treatment of SARS-CoV-2 infected cells.
IC50 (μM): indicates the half maximal concentration able to recover the cytopathic effect of infection and is a measure of the potency of a substance in inhibiting viral included cell death. It was determined from dose-response curves obtained testing the compounds antiviral effect at 8 different concentrations.

The results obtained for the compounds tested are shown in Table 5 below.

All the test compounds are able to inhibit virus replication and reduce cytopathic effects of the virus compared to the control, in particular, they recover cell growth at least to 30% of cell confluence. Interestingly, 50 of the 83 selected compounds (60%) are able to recover more than 50% of cell confluence.

TABLE 5
IC50 and maximum confluence values

| | | Maximum |
|---|---|---|
| Compound | | confluence |
| (current) | IC50 | reached |
| Name | (μM) | (%) |
| Idarubicin hydrochloride | 58.94 | 92.62 |
| Halofantrine hydrochloride | 5.31 | 51.82 |
| 3′-fluorobenzylspiperone | 7.66 | 43.04 |
| IPAG | 12.76 | 81.85 |
| Masitinib (AB1010) | 11.9 | 57.11 |
| thioguanosine | 15.59 | 51.54 |
| CYCLOHEXIMIDE | 1.16 | 63.7 |
| PD153035 | 13.37 | 54.45 |
| GW3965 | 27.71 | 38.01 |
| casin | 14.15 | 50.81 |
| PF-2545920 | 25.26 | 36.73 |
| PPT | 8.3 | 48.2 |
| Endoxifen Hydrochloride | 6.34 | 35.68 |
| Salinomycin | 0.38 | 34.36 |
| Ciclesonide | 46.95 | 35.2 |
| CGP-71683 | 7.36 | 39.25 |

### Example 5: Calu-3 Infection Assay and Spike Protein Immunostaining

Calu-3 cells were seeded in 96-well plates and incubated until they reached full confluence. The cells were subsequently treated with serial dilutions of the investigated compounds (in triplicates per concentration) for 72 h before infection in MEM-Medium containing 10% FCS. Following this pre-incubation, the cells were treated with the same compound dilutions in MEM-Medium containing 1 FCS and infected with SARS-CoV-2 at an MOI of 0.01 for 24 h in the presence of the compounds. After the 24 h incubation, the medium was removed and the cells fixed with Acetone/Methanol-mixture (40:60) for 10 min before blocking with blocking solution (2% BSA, 5% goat serum, 0.01% thimerosal) over night at 4° C.

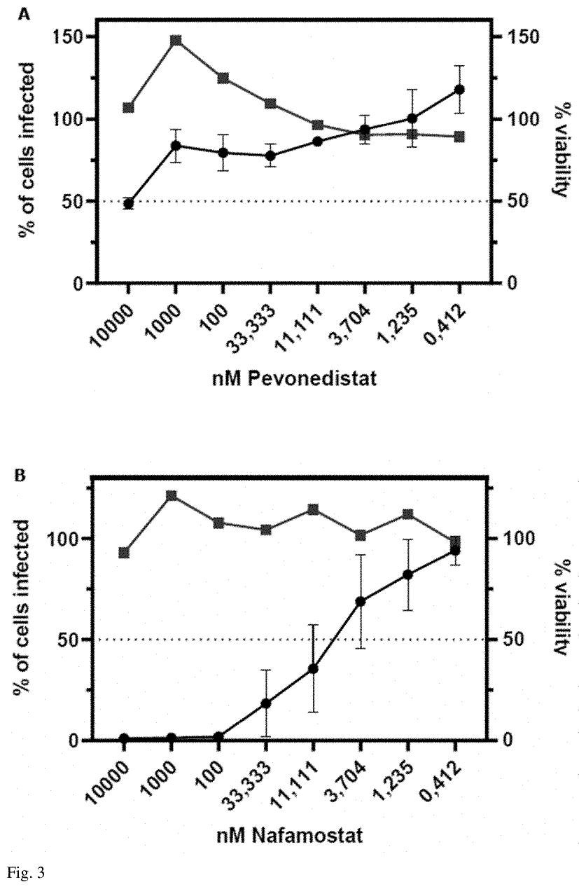

Fixed and blocked cells were incubated with an anti-SARS-CoV-2 spike antibody (rabbit, 1:1500, SinoBiological (Eschborn, Germany)) for 1 h at 37° C., washed twice and subsequently incubated with an HRP-coupled anti-rabbit antibody (goat, 1:1000, Jackson Immunoresearch (Cambridgeshire, UK)) for 1 h at 37° C. After another two washing steps, the cells were stained by addition of 3-Amino-9-ethylcarbazole (AEC) solution for 10 minutes, washed and the percentage of spike positive area was detected in the BIOREADER-7000 F-z device. The percentage of spike positive area per well was quantified and the values of the compound treated samples were normalized to the virus control without compounds (=100%). Values lower or higher than 100% represent virus inhibition or promotion, respectively. Results are shown in FIG. 2; comparative results with compounds described earlier for treatment of infection are shown in FIG. 3. % values are % cells infected compared to an infection control.

### Example 6: Calu-3 Cell Viability Assay

To determine the viability of Calu-3 cells, an MTT assay was performed in parallel at the same compound dilutions as the infection assay but without addition of SARS-CoV-2. The infection assay and the cytotoxicity assay were stopped at the same time and, 25 μl of MTT-Solution was added to the cytotoxicity plates and incubated at 37° C. for 4 h. 100 μl of acidified sodium dodecyl sulfate (SDS) (870 mM, pH 4.7 in water:DMF (50:50)) was added and plates incubated overnight at 37° C. Absorbance at 560 nm with a reference wavelength of 620 nm was measured using a TECAN GENios Basic. To calculate the cell viability, all absorbance values were blank subtracted, the values of untreated cells were set to 100% and the compound-treated samples were correlated to them. Results are shown in FIG. 2; comparative results with compounds described earlier for treatment of infection are shown in FIG. 3. Viability values are given in 100% normalized to a viability control.

### LITERATURE

Bojkova et al. (2020), General Cell Biology & Physiology, doi: 10.21203/rs.3.rs-17218/v1
Corsello et al. (2017), Nature Medicine, 23:405-408, doi:10.1038/nm.4306
Gordon et al. (2020), doi.org/10.1101/2020.03.22.002386v1
Kupferschmidt & Cohen (2020), Science 367:1412, doi: 10.1126/science.367.6485.1412
Li & De Clercq (2020), Nat Rev Drug Discovery 19: 149
Vicar et al. (2019), BMC Bioinformatics, doi: 10.1186/s12859-019-2880-8

TABLE 4
LiGen docking score results

| Compound | | N- | NSP | NSP | NSP | NSP | NSP | NSP | NSP | NSP | | Spike- | |
|---|---|---|---|---|---|---|---|---|---|---|---|---|---|
| Name | 3CL | prot | 3 | 6 | 9 | 12 | 13 | 14 | 15 | 16 | Plpro | ACE2 | Total_score |
| Idarubicin hydrochloride | 5.26 | 4.60 | 5.09 | 3.76 | 4.50 | 4.64 | 4.67 | 5.77 | 4.45 | 4.63 | 4.63 | 5.73 | 57.73 |
| Halofantrine hydrochloride | 5.43 | 5.07 | 4.88 | 4.79 | 4.70 | 4.61 | 4.68 | 5.10 | 5.26 | 5.84 | 4.90 | 5.14 | 60.40 |
| 3′-fluorobenzylspiperone | 5.33 | 5.28 | 5.46 | 4.97 | 4.07 | 4.27 | 4.24 | 5.35 | 4.55 | 4.55 | 5.43 | 6.23 | 59.72 |
| IPAG | 4.65 | 5.15 | 5.00 | 4.38 | 4.53 | 5.02 | 4.76 | 6.09 | 5.07 | 5.28 | 5.18 | 5.14 | 60.24 |
| Masitinib (AB1010) | 5.85 | 5.69 | 4.91 | 5.01 | 5.04 | 5.03 | 4.46 | 6.86 | 4.85 | 5.00 | 5.02 | 6.66 | 64.37 |
| thioguanosine | 4.12 | 4.27 | 4.31 | 5.59 | 4.79 | 4.27 | 4.64 | 5.16 | 4.90 | 5.25 | 4.52 | 5.10 | 56.91 |
| CYCLOHEXIMIDE | 4.29 | 4.38 | 4.97 | 5.02 | 4.64 | 4.56 | 4.26 | 5.54 | 5.37 | 4.84 | 5.23 | 4.45 | 57.54 |
| PD153035 | 4.58 | 4.84 | 5.22 | 5.35 | 3.87 | 4.71 | 5.15 | 6.26 | 4.77 | 4.76 | 5.03 | 4.51 | 59.04 |
| GW3965 | 4.68 | 5.00 | 4.44 | 4.63 | 4.23 | 4.26 | 4.63 | 5.00 | 5.43 | 5.38 | 4.90 | 4.83 | 57.41 |
| casin | 4.90 | 4.46 | 5.17 | 4.46 | 4.61 | 4.26 | 4.38 | 4.94 | 4.40 | 5.75 | 4.49 | 4.85 | 56.68 |
| PF-2545920 | 5.64 | 5.00 | 5.34 | 4.98 | 5.36 | 4.66 | 4.70 | 6.57 | 4.86 | 5.59 | 5.27 | 4.99 | 62.96 |
| PPT | 4.93 | 5.11 | 5.99 | 4.88 | 4.35 | 5.17 | 5.01 | 7.10 | 5.04 | 4.73 | 5.81 | 5.46 | 63.59 |
| Endoxifen Hydrochloride | 4.90 | 4.96 | 6.03 | 5.36 | 4.31 | 4.80 | 4.46 | 5.71 | 5.61 | 5.27 | 4.72 | 5.12 | 61.25 |
| Salinomycin | 4.74 | 5.34 | 4.70 | 3.99 | 5.23 | 5.19 | 4.48 | 3.95 | 4.19 | 4.84 | 5.83 | 6.91 | 59.40 |
| Ciclesonide | 4.63 | 5.11 | 4.63 | 5.95 | 5.00 | 4.64 | 4.73 | 7.21 | 5.21 | 4.52 | 5.69 | 5.57 | 62.88 |
| CGP-71683 | 6.04 | 4.79 | 5.22 | 4.33 | 4.63 | 5.04 | 4.27 | 4.94 | 5.20 | 5.70 | 5.19 | 5.00 | 60.34 |
| Triclocarban | 5.06 | 5.53 | 5.24 | 5.29 | 4.62 | 5.04 | 5.11 | 5.82 | 5.53 | 5.96 | 4.89 | 5.53 | 63.63 |
| Amodiaquine hydrochloride | 4.82 | 4.83 | 4.91 | 4.74 | 4.92 | 4.65 | 4.95 | 6.11 | 5.08 | 4.70 | 5.39 | 5.89 | 61.01 |
| PD-161570 | 5.18 | 5.51 | 5.41 | 5.40 | 5.37 | 5.29 | 4.30 | 5.21 | 4.76 | 4.50 | 4.78 | 5.70 | 61.41 |
| Loperamide Hydrochloride | 4.66 | 4.93 | 6.11 | 4.29 | 5.00 | 4.87 | 5.06 | 6.89 | 4.84 | 5.17 | 5.24 | 5.09 | 62.15 |

TABLE 5
Further results of Examples 1 and 2; % Inh: % inhibition of virus infection, IC50:
concentration of 50% inhibition of virus infection (μM); pIC50: -log₁₀ of IC50;
CC50: concentration of 50% cytotoxicity (μM); SI: selectivity index (CC50/IC50).

| Name | % Inh | IC50 (μM) | pIC50 | CC50 (uM) | SI | PubchemCID |
|---|---|---|---|---|---|---|
| Amuvatinib (MP-470) | 89.4 | 0.02 ± 0 | 7.70 | 0.06 ± 0.01 | 3 | 11282283 |
| polidocanol | 85.8 | 0.22 ± 0.02 | 6.66 | 10.3 ± 1.62 | 47 | 656641 |
| alvocidib | 87.8 | 0.59 ± 0 | 6.23 | 0.06 ± 0 | 0 | 5287969 |
| CETYLPYRIDINIUM CHLORIDE | 100.0 | 0.62 ± 0.05 | 6.21 | 0.66 ± 0.05 | 1 | 2683 |
| Dapivirine (TMC120) | 108.5 | 0.73 ± 0.1 | 6.14 | 5 ± 0.07 | 7 | 214347 |
| LY2228820 | 111.5 | 0.87 ± 0.02 | 6.06 | 56.2 ± 9.79 | 65 | 11539025 |
| Posaconazole | 106.3 | 1.61 ± 0.4 | 5.79 | 14.64 ± 0.67 | 9 | 468595 |
| CHLORMIDAZOLE | 102.1 | 1.89 ± 0.03 | 5.72 | 0.88 ± 0.82 | 0 | 71821 |
| KETOCONAZOLE | 87.1 | 2.36 ± 0.78 | 5.63 | 28.62 ± 4.97 | 12 | 5702077 |
| Acriflavine hydrochloride | 69.4 | | | | | 452192 |
| ACRIFLAVINIUM HYDROCHLORIDE | 99.5 | | | | | 2016 |
| CLOSANTEL | 80.0 | | | | | 42574 |
| HEXACHLOROPHENE | 92.9 | | | | | 3598 |
| Luminespib | 104.5 | | | | | |
| Salinomycin (Procoxacin) | 90.2 | | | | | |
| NSC319726 | 104.2 | <0.02 | 7.70 | <0.02 | 1 | 5351307 |
| AI-10-49 | 103.8 | 0.19 ± 0.06 | 6.72 | 6.58 ± 9.76 | 35 | 49806644 |
| Tyrphostin AG 879 (AG 879) | 104.2 | 0.2 ± 0.01 | 6.70 | 1.36 ± 0.08 | 7 | |
| VLX600 | 109.8 | 0.24 ± 0.07 | 6.62 | 0.06 ± 0 | 0 | 6413301 |
| GSK2606414 | 109.9 | 0.25 ± 0.06 | 6.60 | 70.35 ± 15.28 | >100 | 53469448 |
| ETHAVERINE HYDROCHLORIDE | 85.0 | 0.64 ± 0.53 | 6.19 | 11.04 ± 1.07 | 17 | 3280 |
| AC1NDSS5 | 131.0 | 0.77 ± 0.11 | 6.11 | 0.89 ± 0.83 | 1 | 4581100 |
| SB-612111 | 146.6 | 0.77 ± 0.29 | 6.11 | 3.67 ± 3.21 | 5 | |
| PAPAVERINE HYDROCHLORIDE | 87.7 | 1.1 ± 0.39 | 5.96 | 4.49 ± 1.12 | 4 | 4680 |
| ZK-93423 | 75.1 | 1.34 ± 0.92 | 5.87 | 0.91 ± 0.13 | 1 | 121926 |
| Almitrine dimesilate | 93.2 | 1.42 ± 0.66 | 5.85 | 102.2 ± 73.63 | 72 | 33887 |
| Sorafenib Tosylate | 78.7 | 1.55 ± 0.63 | 5.81 | 1.17 ± 0.43 | 1 | 216239 |
| Octenidine Dihydrochloride | 77.0 | 1.65 ± 0.54 | 5.78 | 2.4 ± 0.09 | 1 | 51167 |
| Regorafenib (BAY 73-4506) | 91.5 | 1.67 ± 0.3 | 5.78 | 0.97 ± 0.6 | 1 | 11167602 |
| THIMEROSAL | 79.4 | 1.69 ± 0.13 | 5.77 | 13.52 ± 1.23 | 8 | 101639657 |
| hematoporphyrin | 96.8 | 1.85 ± 1.08 | 5.73 | 160.08 ± 139.15 | 87 | |
| LDE225 (NVP-LDE225, Erismodegib) | 93.5 | 10.67 ± 7.19 | 4.97 | 2.26 ± 1.28 | 0 | 24775005 |
| DCPIB | 80.3 | 12.78 ± 11.06 | 4.89 | 24.55 ± 11.69 | 2 | 10071166 |
| MEFLOQUINE HYDROCHLORIDE | 93.3 | 14.15 ± 10.05 | 4.85 | 33.02 ± 7.96 | 2 | 4046 |
| AMG-9810 | 93.7 | 14.97 ± 6.7 | 4.82 | 14.22 ± 17.93 | 1 | 680502 |
| Brexpiprazole | 84.8 | 16.04 ± 5.96 | 4.79 | 50.57 ± 68.65 | 3 | 11978813 |
| CC-223 | 104.4 | 16.78 ± 6.9 | 4.78 | 9.49 ± 2.55 | 1 | |
| CBiPES | 139.6 | 17.05 ± 1.17 | 4.77 | 13.4 ± 6.58 | 1 | |
| lidoflazine | 95.9 | 17.39 ± 0.16 | 4.76 | 31.91 ± 10.99 | 2 | 3926 |
| Loteprednol etabonate | 102.5 | 17.92 ± 0.4 | 4.75 | 49.03 ± 45.94 | 3 | 444025 |
| vatalanib | 85.9 | 18.27 ± 0.18 | 4.74 | 64.18 ± 57.35 | 4 | 151194 |
| Adoprazine | 81.4 | 18.59 ± 0.09 | 4.73 | 75.9 ± 54.24 | 4 | 9887537 |
| PH-797804 | 81.5 | 18.85 ± 2.5 | 4.72 | 19.97 ± 3.1 | 1 | 22049997 |
| Cloconazole HCl | 92.8 | 18.92 ± 0.39 | 4.72 | 33 ± 4.61 | 2 | 2880 |
| BP-897 | 80.4 | 18.95 ± 0.43 | 4.72 | 30.03 ± 30.61 | 2 | 3038495 |
| Harringtonine | 93.6 | 19 ± 0.74 | 4.72 | 0.07 ± 0.06 | 0 | 72331 |
| flunarizine | 79.1 | 19.05 ± 0.47 | 4.72 | 136.76 ± 72.31 | 7 | 941361 |
| Lopinavir (ABT-378) | 56.6 | 19.11 ± 0.47 | 4.72 | 2128.78 ± 3592.46 | >100 | 92727 |
| PF-670462 | 111.6 | 19.23 ± 0.7 | 4.72 | 8.24 ± 7.87 | 0 | 11681588 |
| METHYLENE BLUE | 99.2 | 2.03 ± 0.02 | 5.69 | 7.25 ± 2.93 | 4 | 4139 |
| ravuconazole | 103.3 | 2.76 ± 1.3 | 5.56 | 12.75 ± 0.84 | 5 | 467825 |
| Etifoxine (hydrochloride) | 99.7 | 20.64 ± 12.93 | 4.69 | 43.53 ± 38.68 | 2 | 30768 |
| OXICONAZOLE NITRATE | 83.3 | 20.82 ± 0.45 | 4.68 | 26.36 ± 26.02 | 1 | 5353853 |
| LGK-974 | 105.0 | 3.27 ± 1.94 | 5.49 | 7.61 ± 0.06 | 2 | 46926973 |
| Mibampator | 90.1 | 3.28 ± 2.04 | 5.48 | 8.66 ± 1.75 | 3 | 9889366 |
| Avasimibe (CI-1011) | 91.4 | 3.93 ± 1.76 | 5.41 | 13.71 ± 6.18 | 3 | 166558 |
| homoharringtonine | 153.6 | 4.71 ± 2.41 | 5.33 | 1.72 ± 1.89 | 0 | 285033 |
| Pexidartinib | 96.1 | 5.43 ± 1.01 | 5.27 | 41.53 ± 32.45 | 8 | 25151352 |
| Lonafarnib (SCH66336) | 104.4 | 5.68 ± 0.04 | 5.25 | 10.71 ± 7.39 | 2 | 148195 |
| Avatrombopag | 95.9 | 5.71 ± 0.05 | 5.24 | 20.86 ± 29.46 | 4 | 9852519 |
| APIXABAN | 78.0 | 5.91 ± 0.57 | 5.23 | 18.07 ± 1.67 | 3 | 10182969 |
| IPAG | 180.5 | 5.96 ± 0.57 | 5.22 | 11.48 ± 9.83 | 2 | 4239764 |
| JTE-013 | 105.9 | 5.97 ± 0.15 | 5.22 | 16.45 ± 0.42 | 3 | 10223146 |
| drotaverine | 93.1 | 6.07 ± 0.12 | 5.22 | 9 ± 0.96 | 1 | 6437862 |

TABLE 6
Results of comparative experiments according to Examples 1 and 2; % Inh: % inhibition of virus
infection, IC50: concentration of 50% inhibition of virus infection (μM); pIC50: -log₁₀ of
IC50; CC50: concentration of 50% cytotoxicity (μM); SI: selectivity index (CC50/IC50).

| Name | % Inh | IC50 (μM) | pIC50 | CC50 (μM) | SI | PubchemCID |
|---|---|---|---|---|---|---|
| Camostat Mesilate (FOY-305) | 86.9 | 0.64 ± 0.14 | 6.19 | >20 | >30 | 2536 |
| carboxyamidotriazole | 135.1 | 0.09 ± 0.02 | 7.05 | 0.11 ± 0.04 | 1 | 108144 |
| LORATADINE | 91.4 | 15.13 ± 7.45 | 4.82 | 28.33 ± 14.75 | 2 | 3957 |
| MONENSIN SODIUM | 103.2 | | | | | |
| Nafamostat mesylate | 89.8 | 0.04 ± 0.02 | 7.40 | >20 | >100 | 4413 |
| Pevonedistat hydrochloride | 75.1 | 0.63 ± 0.28 | 6.20 | 222.44 ± 332.15 | >100 | 16720766 |
| TIOGUANINE | 99.9 | 1.71 ± 0.33 | 5.77 | 2.21 ± 1.48 | 1 | 2723601 |

1. A method for treating a coronavirus infection in a subject in need thereof or avoiding onset of a coronavirus infection in a statistically significant portion of subjects in need thereof, comprising administering to the subject or subjects a composition comprising a compound selected from the group consisting of compounds indicated in Table 2 and Table 1.

2. The method of claim 1, wherein said compound is a compound selected from the group consisting of LY2228820, NSC319726, polidocanol, Amuvatinib (MP-470), Tyrphostin AG 879 (AG 879), GSK2606414, AI-10-49, VLX600, Ethaverine, alvocidib, Cycloheximide, Cetylpyridinium, thioguanosine, Dapivirine (TMC120), Papaverine, Tanaproget, Octenidine, Almitrine, Sorafenib, ZK-93423, Thimerosal, Regorafenib (BAY 73-4506), Chlormidazole, ravuconazole, Methylene Blue, Mibampator, homoharringtonine, hematoporphyrin, LGK-974, Posaconazole, Ketoconazole, Nelfinavir, JTE-013, Bentamapimod, R05126766 (CH5126766), Lonafarnib (SCH66336), Apixaban, Pexidartinib, drotaverine, LDE225 (NVP-LDE225, Erismodegib, PFK-015, Avatrombopag, 3′-fluorobenzylspiperone, Etifoxine, Avasimibe (CI-1011), Lopinavir (ABT-378), DCPIB, PH-797804, flunarizine, Loteprednol etabonate, lidoflazine, BP-897, Cloconazole, PF-670462, Oxiconazole, AMG-9810, Brexpiprazole, talmapimod, Adoprazine, CC-223, Harringtonine, Barnidipine HCl, vatalanib, OSI-906 (Linsitinib), Idarubicin, Acrylflavine, Basimglurant, NNC-05-2090, Proflavine, Evacetrapib (LY2484595), LY335979 (Zosuquidar trihydrochloride), prenylamine, Fosaprepitant, Mefloquine, SB-657510, P276-00, PD-102807, dexniguldipine, ABC294640, Cyclosporine, and CGP-71683.

3. The method of claim 1, wherein said coronavirus is a beta-coronavirus.

4. The method of claim 1, wherein said coronavirus is severe acute respiratory syndrome coronavirus (SARS-CoV)-2, SARS-CoV-1, or Middle East respiratory syndrome coronavirus (MERS-CoV).

5. The method of claim 1, wherein said coronavirus is SARS-CoV-2.

6. The composition of claim 1, wherein said subject is a human.

7. The method of claim 1, wherein said treating and/or preventing comprises inhibition of coronavirus replication.

8. The method of claim 1, wherein said treating and/or preventing comprises inhibition of cell lysis by said coronavirus.

9. The method of claim 1, wherein said treating or avoiding onset of a coronavirus infection comprises inhibition of tissue destruction by said coronavirus.

10. The method of claim 1, wherein said composition is a pharmaceutical composition.

11. The method of claim 1, wherein the compound is an inhibitor of coronavirus replication that has an IC50 value of at most 50 μM.

12. The method of claim 1, wherein the composition is provided in a kit comprising comprised in a housing.

13. The method of claim 12, wherein the composition further comprises a diluent and the kit comprises a container for the composition from which the composition is administered.

14. The kit of claim 13, wherein the container is an inhaler.

15. A method for identifying an inhibitor of coronavirus replication, the method comprising:
a) contacting human colon carcinoma cells with a coronavirus in the presence of a candidate inhibitor of coronavirus replication,
b) determining virus replication in said human colon carcinoma cells a), wherein said determining virus replication comprises determining proliferation and/or viability of said human colon cancer cells by digital processing of pictures of unlabeled cells and/or quantification of stained nuclei,
c) comparing virus replication determined in b) to a reference, and
d) identifying an inhibitor of coronavirus replication based on a result of the comparing performed in step c).

16. The method of claim 15, wherein said human colon carcinoma cells are CaCo-2 cells.

17. The method of claim 9, wherein said inhibition of tissue destruction comprises inhibition of lung tissue destruction.

18. The method of claim 11, wherein the inhibitor of coronavirus replication has an IC50 value of at most 25 μM.

19. The method of claim 11, wherein the inhibitor of coronavirus replication has an IC50 value of at most 20 μM.

20. The method of claim 14, wherein the composition is administered as an aerosol.
